(19) United States
(12) Patent Application Publication
(10) Pub. No.: US 2026/0325872 A1
(43) Pub. Date: Oct. 22, 2026

(54) ADAPTIVE RADIOTHERAPY CLINICAL DECISION SUPPORT TOOL AND RELATED METHODS

(71) Applicants: H. LEE MOFFITT CANCER CENTER AND RESEARCH INSTITUTE, INC., Tampa, FL (US); THE REGENTS OF THE UNIVERSITY OF MICHIGAN, Ann Arbor, MI (US)

(72) Inventors: Randall K. TEN HAKEN, Ann Arbor, MI (US); Wenbo SUN, Ann Arbor, MI (US); Jionghua JIN, Ann Arbor, MI (US); Ivo D. DINOV, Ann Arbor, MI (US); Kyle Clifford CUNEO, Ann Arbor, MI (US); Martha M. MATUSZAK, Bloomfield Hills, MI (US); Jamalina JAMALUDDIN, Ann Arbor, MI (US); Dipesh NIRAULA, Port Richey, FL (US); Issam EL NAQA, Tampa, FL (US)

(21) Appl. No.: 19/114,138

(22) PCT Filed: Sep. 25, 2023
(86) PCT No.: PCT/US2023/075004

§ 371 Date: Mar. 21, 2025

Related U.S. Application Data
(60) Provisional application No. 63/376,797, filed on Sep. 23, 2022.

(51) Int. Cl. A61N 5/10; G06N 3/045; G06N 3/0464; G06N 3/047; G06N 3/048; G06N 3/09; G06N 3/092; G16H 20/40

(52) U.S. Cl. CPC A61N 5/1031; G06N 3/045; G06N 3/0464; G06N 3/047; G06N 3/048; G06N 3/09; G06N 3/092; G16H 20/40

(57) ABSTRACT

Embodiments of the present disclosure provide methods for training an artificial radiotherapy environment model. The method can include: providing an artificial radiotherapy environment model including: a transition function model configured to predict a next state based on a given state and a given radiation dose for a patient, a radiotherapy outcome estimator model configured to predict a treatment outcome for the next state, the radiotherapy outcome estimator model including at least two artificial neural networks and a logistic function, wherein respective outputs of the at least two artificial neural networks are fed into the logistic function, and a reward function configured to assign a reward for the next state based on the treatment outcome for the next state; and training the artificial radiotherapy environment model with a labeled dataset including a plurality of patient records.

### CROSS-REFERENCE TO RELATED APPLICATIONS

This application claims the benefit of U.S. provisional patent application No. 63/376,797, filed on Sep. 23, 2022, and titled “ADAPTIVE RADIOTHERAPY CLINICAL DECISION SUPPORT TOOL AND RELATED METHODS,” the contents of which are expressly incorporated herein by reference in their entirety.

### STATEMENT REGARDING FEDERALLY FUNDED RESEARCH

This invention was made with government support under Grant no. CA233487 awarded by the National Institutes of Health. The government has certain rights in the invention.

### BACKGROUND

RT efficacy can be improved by personalizing RT treatment. One way of personalizing RT is by following knowledge-based response-adapted RT treatment (KB-ART). In KB-ART, patients' treatment regimen is divided into three phases: Pre-Treatment Assessment, Treatment Response Evaluation, and Treatment Adaptation. In the pre-treatment phase, patients' disease and condition is assessed and a treatment plan is tailored. In the evaluation phase, patients' treatment response is evaluated. Based on their treatment response, associated outcome probabilities are estimated. In the adaptation phase, treatment planning is adapted. The goal of the adaptation is to optimize the treatment outcome, i.e., maximize local tumor control and minimize any radiation induced complication.

### SUMMARY

An Adaptive Radiotherapy Clinical Decision Support (ARCIiDS) software tool is described herein. ARCIiDS is an artificial intelligence-driven clinical decision support tool designed to assist physicians in designing an effective radiotherapy (RT) treatment plan and optimize decision making against cancer. The ARCIiDS tool uses machine learning algorithms in modeling RT environment and for learning and estimating patient's treatment response and treatment outcome. Additionally, the ARCIiDS tool uses deep reinforcement learning (DRL) algorithm in computing optimal dose decision. By using DRL and deep learning (DL), the ARCIiDS tool can recommend optimal prescription adjustment. In some implementations, statistical Ensembling is applied for uncertainty estimate of RT environment prediction and optimal dose recommendation.

An example supervised machine learning model training method is provided. The method includes: providing an artificial radiotherapy environment model including: a transition function model configured to predict a next state based on a given state and a given radiation dose for a patient, a radiotherapy outcome estimator model configured to predict a treatment outcome for the next state, the radiotherapy outcome estimator model including at least two artificial neural networks and a logistic function, wherein respective outputs of the at least two artificial neural networks are fed into the logistic function, and a reward function configured to assign a reward for the next state based on the treatment outcome for the next state; and training the artificial radiotherapy environment model with a labeled dataset including a plurality of patient records, each patient record including respective patient information and a respective retrospective dose plan, wherein the trained artificial radiotherapy environment model is configured to output, for the given state and the given radiation dose, the next state, the treatment outcome for the next state, and the reward for the next state.

In some implementations, the method further includes imposing prior knowledge on at least one feature of the next state for the patient as output by the transition function of the transition function model using a model for radiotherapy.

In some implementations, the model for radiotherapy is a linear-quadraticlinear (LOL) model.

In some implementations, each of the at least two artificial neural networks of the radiotherapy outcome estimator model is tuned individually with the other artificial neural networks of the at least two artificial neural networks fixed.

In some implementations, each of the at least two artificial neural networks of the radiotherapy outcome estimator model is a graph convolutional neural network (GNN).

In some implementations, the radiotherapy outcome estimator model is a generalized logistic function guided double graph convolutional neural network.

In some implementations, the reward function is a function of tumor control probability and normal tissue complication probability where optimizing the reward function maximizes the tumor control probability while minimizing the normal tissue complication probability.

In some implementations, the given state and the next state include one or more multi-omic features.

In some implementations, the given state and the next state include one or more of genomic, radiomic, proteomic, dosimetric, and metabolic tumor volume features.

In some implementations, the treatment outcome for the next state includes a probability of tumor local control and a probability of radiation-induced normal tissue complication.

In some implementations, a reinforcement learning model training method is provided. The method includes: providing a optimal decision-maker model, wherein the optimal decision-maker model includes a deep reinforcement learning model; and training, using the trained artificial radiotherapy environment model, the deep reinforcement learning model, wherein the trained deep reinforcement learning model is configured to predict an optimal dose for the patient.

In some implementations, the optimal dose for the patient maximizes tumor local control and/or minimizes radiation-induced normal tissue complications.

In some implementations, the deep reinforcement learning model is a doubleQ learning model.

In some implementations, the double-Q learning model is trained using a planning and learning scheme.

In some implementations, a method for providing adaptive radiotherapy clinical decision support is provided. The method includes: providing the trained deep reinforcement learning model; receiving a current state for a new patient; inputting the current state into the trained deep reinforcement learning model; and predicting, using the trained deep reinforcement learning model, an optimal treatment dose for the new patient.

In some implementations, the method further includes providing the optimal treatment dose for the new patient.

In some implementations, the method further includes providing an uncertainty estimate.

In some implementations, the uncertainty estimate is related to an output of the trained artificial radiotherapy environment model.

In some implementations, the uncertainty estimate is based on a statistical ensemble.

In some implementations, the uncertainty estimate is related to an output of the trained deep reinforcement learning model.

In some implementations, the uncertainty estimate is based on a statistical ensemble.

In some implementations, the method further includes collecting confidence data related to the trained deep reinforcement learning model from a plurality of users.

In some implementations, the method further includes providing the confidence data.

In some implementations, the confidence data is collected during blind or seen interactions with the plurality of users.

It should be understood that the above-described subject matter may also be implemented as a computer-controlled apparatus, a computer process, a computing system, or an article of manufacture, such as a computer-readable storage medium.

Other systems, methods, features and/or advantages will be or may become apparent to one with skill in the art upon examination of the following drawings and detailed description. It is intended that all such additional systems, methods, features and/or advantages be included within this description and be protected by the accompanying claims.

### BRIEF DESCRIPTION OF THE DRAWINGS

The components in the drawings are not necessarily to scale relative to each other. Like reference numerals designate corresponding parts throughout the several views.

FIG. 1 is a schematic diagram depicting an example Knowledge Based Response-Adaptive Radiotherapy (KBR-ART) assessment workflow.

FIG. 2A is a diagram illustrating example operations for the Adaptive Radiotherapy Clinical Decision Support (ARCIiDS) tool in operation mode (top) and training mode (bottom) according to implementations described herein. ARCIiDS estimates RT treatment outcome via Artificial Radiotherapy Environment (ARTE) and recommends optimal adaptation dose via Optimal Dose Decision-Maker. ARTE is trained first via supervised learning using patient's information and retrospective dose plan as input and corresponding outcome as label. Then, the Optimal Dose Decision-Maker is trained via reinforcement learning with the help of the ARTE. The Optimal Dose Decision-Maker sends patient information and a range of dose to the ARTE and obtains corresponding treatment outcomes for training purposes.

FIG. 2B is a diagram illustrating example operations for training the artificial radiotherapy environment and optimal dose decision-maker modules of the ARCIiDS tool according to implementations described herein.

FIG. 3 is an example computing device.

FIG. 4A is a graph depicting a transition function for generalized equivalent uniform dose (gEUD).

FIG. 4B is a table showing a Transition Function for final gEUD.

FIG. 5A shows example Radio Therapy Outcome Estimators (RTOE).

FIG. 5B is a is a diagram illustrating example operations for training RTOE as a binary classification.

FIG. 5C is a directed graph showing the inter-relation between the nonsmall cell lung carcinoma (NSCLC) patient's features.

FIG. 5D is a directed graph showing the inter-relation between the HCC patient's features.

FIG. 6 is a Reward Function 3D Contour Plot.

FIG. 7 shows Model Uncertainty via statistical Ensemble.

FIG. 8 is a self-evaluation scheme for AI recommendation.

FIG. 9 depicts an ARCIiDS graphical user interface (GUI).

FIG. 10A, FIG. 10B, and FIG. 10c are graphs showing a comparison and analysis of two ARCIiDS models trained and validated on Adaptive radiotherapy (RT) nonsmall cell lung carcinoma (NSCLC) patients.

FIG. 11A, FIG. 11B, and FIG. 11C are graphs showing comparison and analysis of two ARCIiDS models trained and validated on Adaptive radiotherapy (RT) hepatocellular carcinoma (HCC) patients.

### DETAILED DESCRIPTION

Unless defined otherwise, all technical and scientific terms used herein have the same meaning as commonly understood by one of ordinary skill in the art. Methods and materials similar or equivalent to those described herein can be used in the practice or testing of the present disclosure. As used in the specification, and in the appended claims, the singular forms “a,” “an,” “the” include plural referents unless the context clearly dictates otherwise. The term “comprising” and variations thereof as used herein is used synonymously with the term “including” and variations thereof and are open, non-limiting terms. The terms “optional” or “optionally” used herein mean that the subsequently described feature, event or circumstance may or may not occur, and that the description includes instances where said feature, event or circumstance occurs and instances where it does not. Ranges may be expressed herein as from “about” one particular value, and/or to “about” another particular value. When such a range is expressed, an aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

As used herein, the terms “about” or “approximately” when referring to a measurable value such as an amount, a percentage, and the like, is meant to encompass variations of +20%, +10%, +5%, or +1% from the measurable value.

“Administration” of “administering” to a subject includes any route of introducing or delivering to a subject an agent. Administration can be carried out by any suitable means for delivering the agent. Administration includes self-administration and the administration by another.

The term “subject” is defined herein to include animals such as mammals, including, but not limited to, primates (e.g., humans), cows, sheep, goats, horses, dogs, cats, rabbits, rats, mice, and the like. In some embodiments, the subject is a human.

The adaptive radiotherapy clinical decision support methods and systems described herein can be used to predict an optimal radiation dose for treating a solid tumor in a subject (or patient). Optionally, the optimal radiation dose is used for treatment plan adaptation during the latter stages of RT. A solid tumor is an abnormal mass of hyperproliferative or neoplastic cells from a tissue other than blood, bone marrow, or the lymphatic system, which may be benign or cancerous. In general, the tumors described herein are cancerous. As used herein, the terms “hyperproliferative” and “neoplastic” refer to cells having the capacity for autonomous growth, i.e., an abnormal state or condition characterized by rapidly proliferating cell growth. Hyperproliferative and neoplastic disease states may be categorized as pathologic, i.e., characterizing or constituting a disease state, or may be categorized as non-pathologic, i.e., a deviation from normal but not associated with a disease state. The term is meant to include all types of solid cancerous growths, metastatic tissues or malignantly transformed cells, tissues, or organs, irrespective of histopathologic type or stage of invasiveness. “Pathologic hyperproliferative” cells occur in disease states characterized by malignant tumor growth. Examples of non-pathologic hyperproliferative cells include proliferation of cells associated with wound repair. Examples of solid tumors are sarcomas, carcinomas, and lymphomas. Leukemias (cancers of the blood) generally do not form solid tumors.

The term “carcinoma” is an art recognized term and refers to malignancies of epithelial or endocrine tissues including respiratory system carcinomas, gastrointestinal system carcinomas, genitourinary system carcinomas, testicular carcinomas, breast carcinomas, prostatic carcinomas, endocrine system carcinomas, and melanomas. In the examples described herein, the disease is non-small cell lung carcinoma (NSCLC) (also referred to as “nonsmall cell lung cancer”). It should be understood that non-small cell lung carcinoma is provided only as an example. This disclosure contemplates that the adaptive radiotherapy clinical decision support methods and systems described herein can be used to treat other cancers. For example, the disease may be hepatocellular carcinoma, rectal carcinoma, colon carcinoma, esophageal carcinoma, prostate carcinoma, head and neck carcinoma, or melanoma. Exemplary carcinomas include those forming from tissue of the cervix, lung, liver, prostate, breasₜ, head and neck, colon, and ovary. The term also includes carcinosarcomas, e.g., which include malignant tumors composed of carcinomatous and sarcomatous tissues. An “adenocarcinoma” refers to a carcinoma derived from glandular tissue or in which the tumor cells form recognizable glandular structures.

Optimal decision-making in Knowledge Based Response-Adaptive Radiotherapy (KBR-ART) is a difficult task [1]. The difficulties arise from a slew of factors, such as, involvement of many variables, uncertainty in treatment response, and inter-patient heterogeneity [2]. In the absence of a quantitative framework, clinical decisions are primarily influenced by physician's professional experiences, which may result in inter-physician variability. Thus, there is a need for a robust and user-friendly clinical decision-support tool for objective decision-making in KBR-ART that is data-driven and consistent [3].

Adaptive Radiotherapy Clinical Decision Support (ARCIiDS) is a web-based software tool for AI-assisted optimal decision-making in KBR-ART [4-6] and potentially other oncology applications involving dynamic treatment regime (DTR) [7,8]. ARCIiDS provides a quantitative approach to overcome the decision-making difficulties via a set of data analytics algorithms, which include feature selection of important variables, statistical ensemble for representing uncertainties of treatment response, and, most importantly, integration of information-rich dense multi-omics datasets for capturing inter-patient heterogeneity [9-11]. ARCIiDS combines all the above data analytics capabilities and presents a user-friendly interface for evaluating relevant clinical use cases. Moreover, it is complementary to the current treatment planning system; the integration may facilitate an introduction of multi-omics information into the treatment planning workflow.

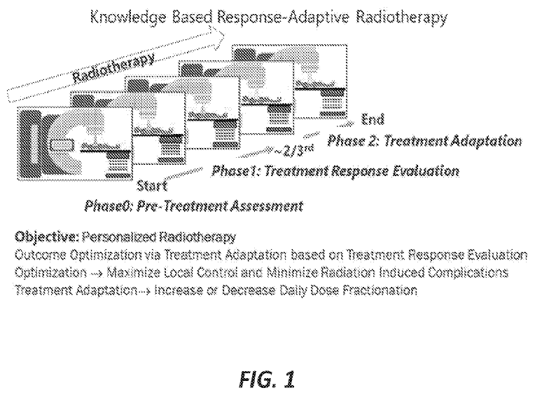

Referring now to FIG. 1, a schematic diagram depicting an example KBR-ART assessment workflow is provided. In KBR-ART, a pre-treatment assessment is conducted in phase 0 and an appropriate treatment plan is tailored. Then patients' treatment response is evaluated in Phase 1, and an optimal treatment adaptation is planned and executed in Phase 2.

DTR including adaptive RT (ART) are designed for treatment personalization. A popular ART paradigm and implementation is to adapt treatment plans to accommodate during-treatment anatomical changes due to weight loss, tumor regression and/or diminution of the volume of surrounding normal tissue and organ at risk (OAR). A complementary ART paradigm is KBR-ART which provides a response-based adaptive framework for personalizing RT as shown in FIG. 1, where the response assessment is not limited to observing anatomical changes. It is divided into three phases: Pre-Treatment Assessment, Treatment Response Evaluation (evaluation phase) and Treatment Adaptation (adaptation phase). In the pre-treatment phase, a patient's disease and condition is assessed and a treatment plan is tailored. In the evaluation phase, a patient's treatment response is evaluated by comparing pre and mid treatment multi-omics information changes. Based on the treatment responses, the patient's associated outcome probabilities are estimated. In the adaptation phase, treatment planning is adapted for a personalized and an optimal outcome. Two endpoints are considered: tumor control and normal tissue complication. The goal of KBRART is to maximize tumor control probability (TCP) and minimize normal tissue complication probability (NTCP).

To demonstrate the potential of ARCIiDS, two clinical use cases are presented. In both studies, the evaluation time was around 1 month. The first use case is based on the UMCC (University of Michigan Cancer Center) 2007-123 phase II dose escalation clinical trial NCT01190527 13, where inoperable or unresectable non-small cell lung cancer (NSCLC) patients were administered with 30 daily dose fractions. The patients received roughly 50 Gy [Gray=J/Kg] equivalent dose in 2 Gy fractions (EQD2) in the evaluation phase and up to a total dose of 92 Gy EQD2 in the adaptation phase. The evaluation phase lasted for roughly two-thirds of the 6-week treatment period. In the second clinical use case, patients with hepatocellular carcinoma (HCC) received adaptive stereotactic Body Radiation Therapy (SBRT) in clinical trials NCT01519219, NCT01522937, and NCT0246083514. In the evaluation phase, patients received 3 daily dose fractions followed by 1 month break, and in the adaptation phase, a suitable subpopulation of the patients received 2 additional daily doses.

A large sample size that is representative of the true population is preferred for all data driven and statistical modeling. However, due to financial, feasibility, and ethical reasons, obtaining a large dataset in the medical field is often impractical. In the instant case, a dataset of size 117 and 292 were available for NSCLC patients and HCC patients, respectively. Dense multi-omics data with 297 features were available for only 67 NSCLC patients and 110 features for 71 HCC patients. These datasets, albeit on the smaller size, are unique as KBR-ART is still in its clinical trial phase and hence the largest multi-omics datasets for KBR-ART.

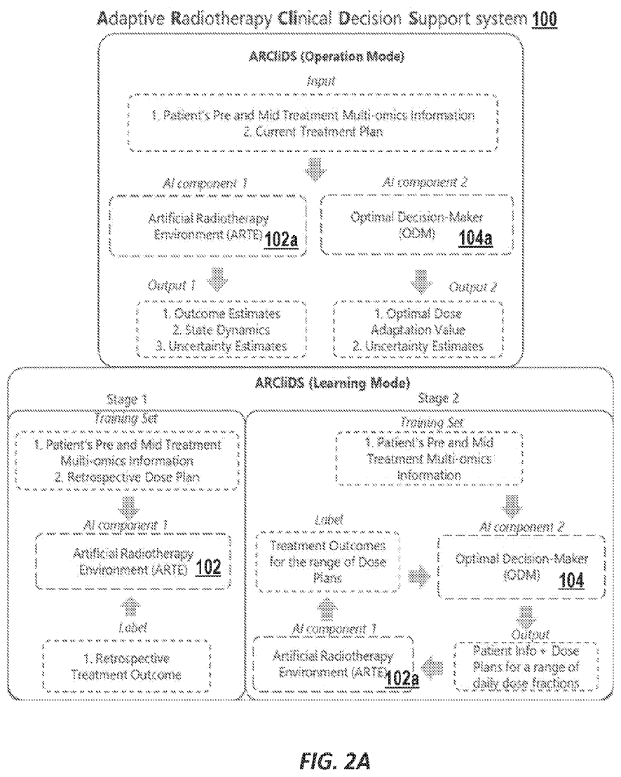

Under the current United states Food and Drug Administration (FDA) definition and guidelines, ARCIiDS is categorized as a Software as a Medical Device (SaMD) [6]. SaMD is defined as software intended to be used for medical purposes independently in contrast to software intended to drive a hardware medical device (software in a medical device). This definition was recently adopted by FDA to include Artificial Intelligence (AI) software [15,16] which can automatically learn from user cases and continuously update after deployment, as opposed to traditional software, which stays fixed after deployment (excluding version update). Therefore, the ARCIiDS system also has two modes of operation: Operation Mode and Learning Mode as shown in FIG. 2A. After the initial training, both modes can run simultaneously (online learning) in the clinic.

ARCIiDS is composed of two main AI components. The first component is the Artificial Radiotherapy Environment (ARTE) for estimating the predicted outcome and the second component is the Optimal Decision-Maker (ODM) for decision-making. In Operation Mode, ARCIiDS asks for a patient's pre and mid treatment multi-omics information, and current treatment plan. It feeds that information into ARTE and ODM, and obtains outcome estimates, state dynamics, and the optimal dose adaptation value. All of the estimated results come with associated uncertainty. The results are presented in two main plots: outcome space spanned by TCP and NTCP, and population distribution plots as further explained in the Graphical User Interface (GUI). During the Learning Mode, ARTE is trained first on the available data, and then ODM is trained on the ARTE. The details of the training are presented herein.

ARCIiDS presents a significant improvement to Tseng et al.'s and Niraula et al.'s [5] methods. The improvement comes from the graphical representation of patients' features. Convolution neural networks (CNNs) are known to perform well because they exploit the feature locality of images [18]. In other words, pixels at neighboring areas of an image are correlated, and CNN architectures can capture those correlations. Graph Neural Networks (GNNs) are similar except they exploit the non-local relationship between feature values [19]. Computationally, GNNs use fewer network connections compared to fully connected NNs, which helps in learning by reducing redundancies. From another perspective, information from one feature only goes to its neighboring features. Embodiments of the present disclosure utilize the feature graph from Luo et al.'s work on multi-objective Bayesian Network which identified the most important features related to RT outcome of interest by finding the Markov Blanket of the outcomes. Details of feature selection procedure are presented herein. For NSCLC and HCC, 13 important features were selected.

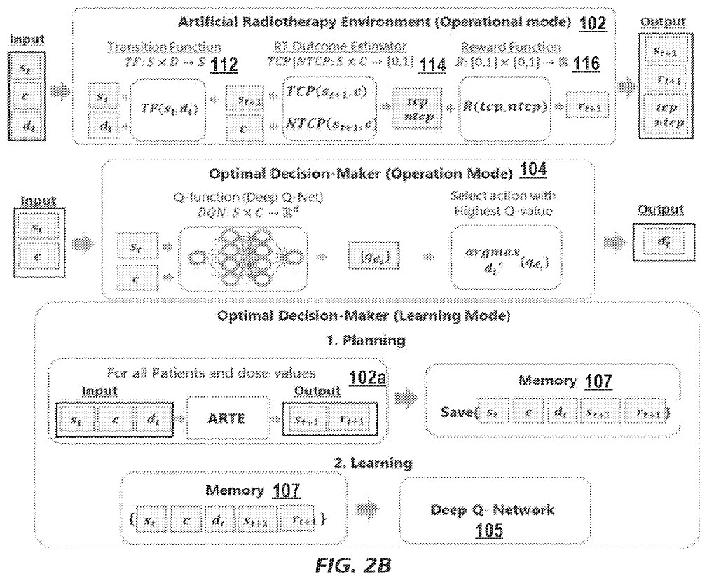

An Adaptive Radiotherapy Clinical Decision Support (ARCIiDS) system 100 and related methods are described below with regard to FIG. 2A, FIG. 2B, and FIG. 3. As shown in FIG. 2A, in operation mode (or inference mode), the ARCIiDS system 100 includes a trained artificial radiotherapy environment model 102a (“AI component 1”) and a trained optimal decisionmaker (ODM) model 104a (“AI component 2”). The untrained models are referred to herein as artificial radiotherapy environment (ARTE) model 102 (“AI component 1”) and optimal decisionmaker (ODM) model 104 (“AI component 2”). As described below, an untrained model is trained with a training set (e.g., data set or dataset) to “learn” a function that maps model input (one or more “features”) to an output (one or more “targets”). In other words, the untrained artificial radiotherapy environment model 102 and optimal decision-maker model 104 are trained with training sets (e.g., datasets), which results in adjusting model parameters such as weights and/or biases, to predict specific targets as described in more detail below. It should be understood that the untrained models may not yield sufficient accuracy for use in operation mode. Thus, the trained models are labeled ARTE model 102a and optimal decision-maker model 104a to indicate that such models are sufficiently accurate and useful for operation mode. As illustrated, during operation mode, the ARCIiDS system 100 uses a patient's pre- and mid-treatment multi-omics information and current treatment plan as an input. In the operation mode, the output of the ARTE model 102a (“Output 1”) can comprise outcome estimates, state dynamics, RT outcome estimates, and/or uncertainty estimates, and the output of the ODM model 104a (“Output 2”) can comprise an optimal dose adaptation value or recommendation and/or uncertainty estimates.

In one implementation, an example supervised machine learning model training method is described. A supervised machine learning model “learns” a function that maps an input (also known as feature or features) to an output (also known as target or targets) during training with a labeled data set. Supervised learning models include, but are not limited to, artificial neural networks, including deep neural networks. An artificial neural network (ANN) is a computing system including a plurality of interconnected neurons (e.g., also referred to as “nodes”). This disclosure contemplates that the nodes can be implemented using a computing device (e.g., a processing unit and memory as described herein). The nodes can be arranged in a plurality of layers such as input layer, output layer, and optionally one or more hidden layers. An ANN having hidden layers can be referred to as deep neural network or multilayer perceptron (MLP). Each node is connected to one or more other nodes in the ANN. For example, each layer is made of a plurality of nodes, where each node is connected to all nodes in the previous layer. The nodes in a given layer are not interconnected with one another, i.e., the nodes in a given layer function independently of one another. As used herein, nodes in the input layer receive data from outside of the ANN, nodes in the hidden layer(s) modify the data between the input and output layers, and nodes in the output layer provide the results. Each node is configured to receive an input, implement an activation function (e.g., binary step, linear, sigmoid, hyperbolic tangent (tanh), or rectified linear unit (ReLU) function), and provide an output in accordance with the activation function. Additionally, each node is associated with a respective weight. ANNs are trained with a data set to minimize the cost function, which is a measure of the ANN's performance (e.g., error such as L1 or L2 loss) during training. The training algorithm tunes the node weights and/or bias to minimize the cost function. This disclosure contemplates that any algorithm that finds the minimum of the cost function can be used for training the ANN. Training algorithms for ANNs include, but are not limited to, backpropagation. It should be understood that artificial neural networks are provided only as example supervised machine learning models.

In some implementations, the ARCIiDS system 100 trains the ARTE model 102 via supervised learning. The ARTE model 102a is then utilized in planning and teaching the ODM Model 104 via reinforcement learning. Referring again to FIG. 2A, an example learning mode for the ARCIiDS system 100 can comprise stage 1 and stage 2. During stage 1, the ARTE model 102 is trained with a labeled training set or dataset comprising a plurality of patient records (e.g., respective patient information and a respective retrospective treatment outcome and/or dose plan). In the example shown in FIG. 2A, the training set or dataset includes patient pre- and midtreatment multi-omics information for a plurality of patients and retrospective dose plan(s). During stage 2, the ODM model 104 is trained with a labeled training set or dataset comprising a plurality of patient records (e.g., the same training set or a different training set). In the example shown in FIG. 2A, the training set or dataset includes patient pre- and mid-treatment multi-omics information for a plurality of patients. The output of the trained ARTE model 102a, which comprises treatment outcomes for the range of dose plans, is also used to train the ODM model 104 via reinforcement learning. The ODM model 104 is trained to output patent information and dose plans for a range of daily dose fractions.

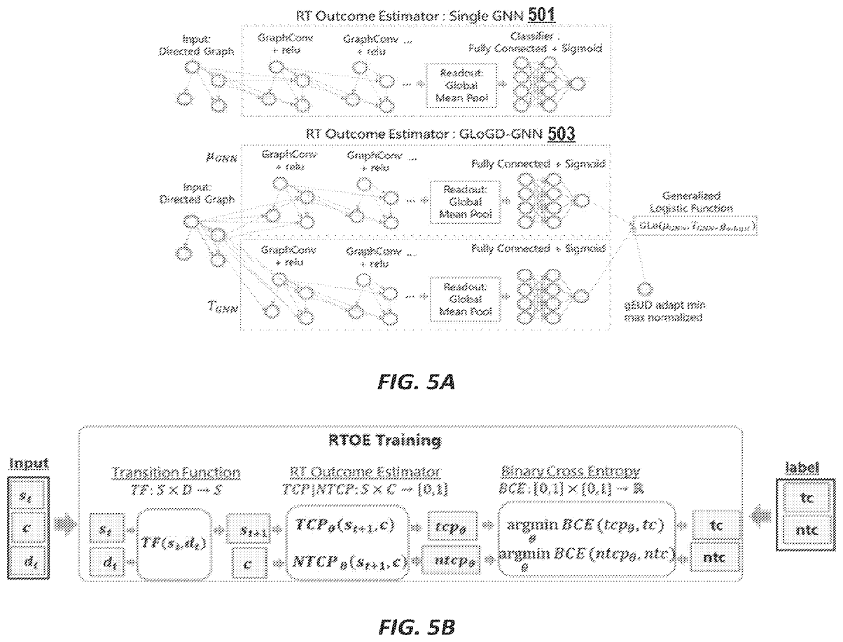

As shown in FIG. 2B, the method includes providing an artificial radiotherapy environment model 102 that includes: a transition function model 112, a radiotherapy outcome estimator model 114, and a reward function 116. In some implementations, the reward function 116 is a contour plot as described in connection with FIG. 6. The transition function model 112 is configured to predict a next state or resulting state (sₜ+1) (used interchangeably herein) based on a given state (sₜ) and a given radiation dose (dₜ) for a patient. Thus, the transition function model 112 features are given state (sₜ) and given radiation dose (dₜ), and the transition function model 112 target is next state (sₜ₊₁). In other words, the transition function model 112 is trained to predict next state (sₜ₊₁) (i.e., target) based on given state (sₜ) and given radiation dose (dₜ) (i.e., features). In some embodiments, the given state and next state comprise one or more multi-omic features, such as, but not limited to genomic, radiomic, proteomic, dosimetric, imaging, clinical, and metabolic tumor volume features. In some implementations, the transition function model 112 is an artificial neural network. An example neural network architecture for the transition function model 112 is provided herein. It should be understood that the neural network architecture described herein is only provided as an example. This disclosure contemplates that the transition function model 112 can be an artificial neural network having a different architecture than described herein. Alternatively, this disclosure contemplates that the transition function model 112 can be implemented with another type of supervised machine learning model.

In some implementations, the given state (sₜ) and the next state (sₜ₊₁) include one or more multi-omic features, which may include, but are not limited to, genomic, radiomic, proteomic, dosimetric, imaging, clinical, metabolic tumor volume (MTV), and cytokine features. Exemplary NSCLC features can include: cytokines: pretreatment interleukin 4 (pre-IL4), pre-IL15 and slope of Interferon gamma-induced protein 10 (slope-IP10); Tumor PET imaging features/Radiomics: pretreatment Metabolic Tumor Volume (pre-MTV), relative difference (RD) of Gray-level size zone matrices (GLSZM)-large zone low gray-level (LZLGE) and RD-GLSZM-zone size variance (RD-GLSZM-ZSV); Dosimetry: Tumor gEUD and Lung gEUD; Genetics (single nucleotide polymorphism [SNP]): Cxcr1-Rs2234671, Ercc2-Rs238406, and Ercc5-Rs1047768; and MicroRNA: miR-191-5p and miR-20a-5p. Exemplary HCC features can include: clinical: sex, age, pretreatment cirrhosis status (pre-cirrhosis), pretreatment Eastern Cooperative Oncology Group Performance status (pre-ECOG-PS), number of active liver lesions (active lesions), pretreatment albumin level (pre-albumin); Tumor PET Imaging: gross tumor volume (GTV) and liver volume minus GTV (Liver-GTV); Dosimetry: GTV gEUD and Liver-GTV volume; and cytokines/signaling molecule: relative difference of Transforming growth factor beta (RD-TGFβ), Cluster of Differentiation 40 receptor's Ligand (RD-CG40L), and Hepatocyte growth factor (RD-HGF). In the above examples, the slope and RD can be determined by comparing pretreatment and mid-treatment or the end of evaluation phase.

It should be understood that the multi-omic features provided above are only provided as examples. This disclosure contemplates using different types and/or greater or less state features than provided as examples.

Optionally, in some implementations, the method further includes imposing prior knowledge on at least one feature of the next state (sₜ₊₁) for the patient, which is as output by the transfer function model 112 and using a model for radiotherapy. In some implementations, the model for radiotherapy is a linear-quadratic-linear model. For example, prior knowledge can be imposed on organ (e.g., lung) and tumor generalized equivalent uniform dose (gEUD) using a linear quadratic model for radiotherapy. gEUD is a measure of radiation absorbed (e.g., by the organ, tumor) and is expected to increase (decrease) with increasing (decreasing) radiation delivered to the patient. Use of the linear-quadratic-linear model to impose prior knowledge on gEUD is described herein.

As depicted in FIG. 2B, the radiotherapy outcome estimator model 114 estimates tumor control probability (tcp) and normal tissue complication probability (ntcp) for a patient in state sₜ₊₁ and covariate c. The reward function R assigns a reward rₜ₊₁ to the tuple (tcp, ntcp), so that optimal reward corresponds to maximal tcp and minimal ntcp. Overall, given sₜ, c, and dt, ARTE model 102 yields sₜ₊₁, rₜ₊₁, tcp and ntcp. The radiotherapy outcome estimator model 114 is configured to predict a treatment outcome for the next state (sₜ₊₁). Thus, the radiotherapy outcome estimator model 114 feature is the next state (sₜ₊₁), which is predicted by the transition function model 112, and the radiotherapy outcome estimator model 114 target is treatment outcome. Treatment outcome can include, but is not limited to, tumor control probability (tcp) (sometimes also referred to as probability of tumor local control (Pic)) and normal tissue complication probability (ntcp) (sometimes also referred to as probability of radiation-induced complication (PRNTC)). It should be understood that tcp and ntcp are provided only as examples for treatment outcome. In other words, the radiotherapy outcome estimator model 114 is trained to predict tumor control probability (tcp) and normal tissue complication probability (ntcp) (i.e., targets) based on the next state (sₜ₊₁) (i.e., feature). In the examples described herein, the ARCIiDS tool is used to treat lung cancer and liver cancer. In the example of lung cancer, local control refers to disappearance of a lung tumor (i.e., primary tumor) and radiation-induced complication is radiation-induced pneumonitis. As described above, lung cancer is provided only as an example disease. This disclosure contemplates that local control can refer to disappearance of a primary tumor in organs other than the lung (e.g., the liver) and that radiation-induced complication can refer to conditions other than pneumonitis, which is specific to the lung. The ARCIiDS tool is also applicable to different treatment types. For example, the ARCIiDS tool can be used to treat lung cancer with traditional RT (e.g., 50-60 Gy over 30 fractions). In another example, the ARCIiDS tool is used to treat liver cancer with SBRT (e.g., 50-60 Gy over 5 fractions, which is a higher dose/fraction that that used to treat lung cancer).

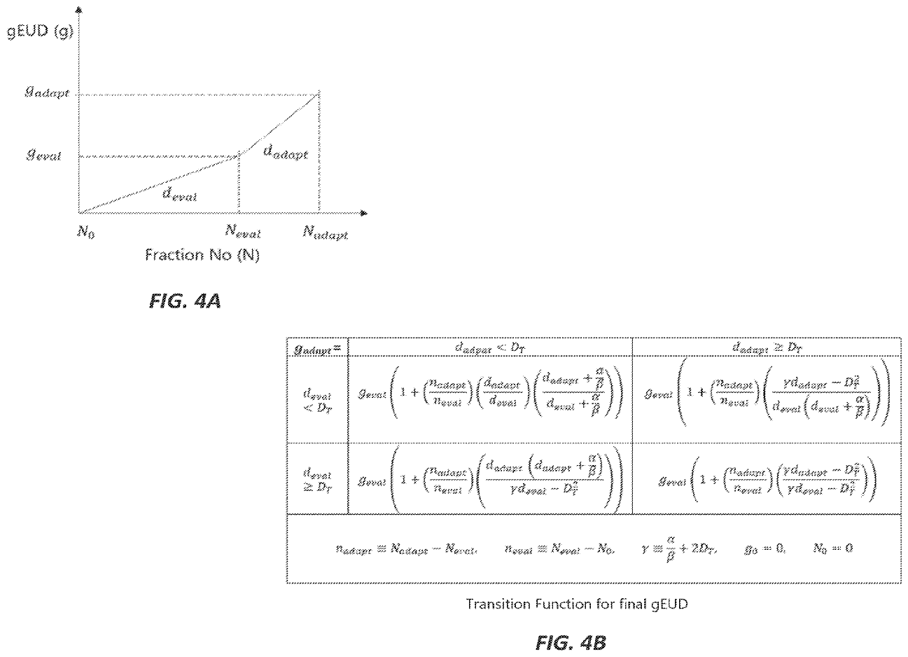

In some implementations, the radiotherapy outcome estimator model 114 includes at least two artificial neural networks. Each of the at least two artificial neural networks of the radiotherapy outcome estimator model can be at least one graph convolutional neural network (GNN). Optionally, in some implementations, each of the at least two artificial neural networks of the radiotherapy outcome estimator model 114 is tuned individually with the other artificial neural networks of the at least two artificial neural networks fixed. Example neural network architectures for the radiotherapy outcome estimator model 114 are provided herein. It should be understood that the neural network architectures described herein are only provided as examples. This disclosure contemplates that the radiotherapy outcome estimator model 114 can be artificial neural networks having a different architecture than described herein. Alternatively, this disclosure contemplates that the radiotherapy estimator model 114 can be implemented with another type of supervised machine learning model. Additionally, the radiotherapy outcome estimator model 114 includes a logistic function, where respective outputs of the at least two artificial neural networks are fed into the logistic function. As described herein, the logistic function imposes prior knowledge on the outputs of the at least two artificial networks such that probability of tumor local control (P_{LC}) and probability of radiation-induced complication (PRNTC) respond appropriately (increase/decrease) to the administered radiation. In particular, Pic should increase (decrease) with increasing (decreasing) radiation, and PRNTC should also increase (decrease) with increasing (decreasing) radiation. The logistic function ensures such monotonic property. In some implementations, a Graph Convolution Neural Network is used as a classifier as discussed with reference to FIG. 5A below. The Graph Convolution Neural Network can read features as a graph instead of a vector (e.g., a 13×1 one-dimensional vector)

The reward function 116 is configured to assign a reward (rₜ₊₁) for the resulting state based on the treatment outcome (tcp and ntcp) for the next state. Thus, the reward function 116 inputs are the treatment outcome (tcp and ntcp) for the next state, which is predicted by the radiotherapy outcome estimator model 114, and the reward function 116 output is the reward (rₜ₊₁) for the next state. In other words, the reward function 116 assigns a reward (i.e., output) for the next state based on the treatment outcome (tcp and ntcp) for the next state (i.e., inputs). In some implementations, the reward function is a function of tumor control probability and normal tissue complication probability where optimizing the reward function maximizes tumor control probability while minimizing normal tissue complication probability. In some examples, the reward function can be depicted as a contour plot. An example contour plot for the reward function 116 is provided herein. It should be understood that the contour plot described herein is only provided as an example. This disclosure contemplates that the reward function 116 can be a contour plot or plots different than described herein.

The method also includes training the artificial radiotherapy environment model 102 with a labeled dataset including a plurality of patient records, each patient record including respective patient information and a respective retrospective dose plan. As described above, supervised machine learning models are trained with a data set minimizing a cost function, which is a measure of the model's performance. Accordingly, as shown in FIG. 2B, the trained artificial radiotherapy environment model 102a is configured to output, for the given state (sₜ) and the given radiation dose (dₜ), the next state (sₜ₊₁), the treatment outcome (tcp and ntcp) for the next state, and the reward (rₜ₊₁) for the next state.

As depicted in FIG. 2B, ODM 104 comprises a deep Q-network (DQN) 105 and decision selector. Given a state (sₜ, c), DQN 105 yields a q (quality) value for the range of adaptive dose. In operation mode, the selector greedily selects the dose with the highest q-value. The ODM 104 is trained by following the model-based reinforcement learning paradigm. In the Planning Phase, the ODM 104 saves next states {sₜ₊₁} and associated rewards {rₜ₊₁} for all patient's state {(sₜ, c)} and the range of adaptive dose {dₜ}. In the Learning phase, a double DQN algorithm is applied on the memory 107.

In another implementation, an example reinforcement learning model training method is described. The method includes providing an ODM model 104 that includes a deep reinforcement learning model. In some implementations, the deep reinforcement learning model is a double-Q learning model. In some embodiments, the double-Q learning model is trained using a planning and learning scheme. An example neural network architecture for the double-Q learning model (Deep Q-Net Architecture) is provided herein. It should be understood that the neural network architecture described herein is only provided as an example. This disclosure contemplates that the double-Q learning model can be an artificial neural network having a different architecture than described herein. Alternatively, this disclosure contemplates that the ODM model 104 can be implemented with another type of deep reinforcement learning model. As shown in FIG. 2A, the method also includes training, using the trained artificial radiotherapy environment model 102a, the ODM model 104. As described above, the trained artificial radiotherapy environment model 102a predicts the next state (sₜ₊₁), the treatment outcome (Pic and PRNTC) for the next state, and the reward (rₜ₊₁) for the next state, which are used to train the ODM 104 (e.g., deep reinforcement learning model or other model). Then, as shown in FIG. 2A, the trained deep reinforcement learning model 104a is configured to predict an optimal dose for the patient. Optionally, the optimal dose is used for dose adaptation during the latter stages of RT delivered to the patient. In some implementations, the optimal dose for the patient maximizes tumor local control. In some implementations, the optimal dose for the patient minimizes radiation-induced complications. In some implementations, the optimal dose for the patient maximizes tumor local control and minimizes radiation-induced complications.

An example method for providing adaptive radiotherapy clinical decision support is also described herein. The method includes providing a trained deep reinforcement learning model, for example, the trained deep reinforcement learning model 104a shown in FIG. 2A. The method also includes receiving a current state for a new patient; inputting the current state into the trained deep reinforcement learning model; and predicting, using the trained deep reinforcement learning model, an optimal treatment dose for the new patient. For example, in some implementations, the trained deep reinforcement learning model takes in patient's current state as input and generates q-values for all possible actions (dose decision) as output. Optimal dose is then selected using greedy-policy, i.e., to select dose with maximum q-value. Optionally, the optimal dose is used for dose adaptation during the latter stages of RT.

Optionally, in some implementations, the method further includes providing the optimal treatment dose for the new patient. For example, the optimal treatment dose can be displayed graphically on a computing device (e.g., computing device shown in FIG. 3). Alternatively or additionally, the optimal treatment dose can be transmitted from a computing device (e.g., computing device shown in FIG. 3) to a user over a communication link. This disclosure contemplates a communication links is any suitable communication link. For example, a communication link may be implemented by any medium that facilitates data exchange including, but not limited to, wired, wireless and optical links. Example communication links include, but are not limited to, a LAN, a WAN, a MAN, Ethernet, the Internet, or any other wired or wireless link such as WiFi, WiMax, 3G, 4G, or 5G.

In some implementations, the method further includes providing an uncertainty estimate. The uncertainty estimate can optionally be graphically displayed and/or transmitted over a communication link to a user as described above. For example, the uncertainty estimate can be related to an output of the trained artificial radiotherapy environment model (see e.g., trained artificial radiotherapy environment model 102a shown in FIGS. 2A and 2B). As described herein, such an uncertainty estimate can optionally be based on a statistical ensemble. Alternatively or additionally, the uncertainty estimate can be related to an output of the trained deep reinforcement learning model, (see e.g., trained deep reinforcement learning model 104a shown in FIG. 2B). As described herein, such an uncertainty estimate can optionally be based on a statistical ensemble.

In some implementations, the method further includes collecting confidence data related to the trained deep reinforcement learning model from a plurality of users. The method optionally further includes providing the confidence data. The confidence data can optionally be graphically displayed and/or transmitted over a communication link to a user as described above. As described herein, such confidence data can be collected during blind or seen interactions with the plurality of users.

It should be appreciated that the logical operations described herein with respect to the various figures may be implemented (1) as a sequence of computer implemented acts or program modules (i.e., software) running on a computing device (e.g., the computing device described in FIG. 3), (2) as interconnected machine logic circuits or circuit modules (i.e., hardware) within the computing device and/or (3) a combination of software and hardware of the computing device. Thus, the logical operations discussed herein are not limited to any specific combination of hardware and software. The implementation is a matter of choice dependent on the performance and other requirements of the computing device. Accordingly, the logical operations described herein are referred to variously as operations, structural devices, acts, or modules. These operations, structural devices, acts and modules may be implemented in software, in firmware, in special purpose digital logic, and any combination thereof. It should also be appreciated that more or fewer operations may be performed than shown in the figures and described herein. These operations may also be performed in a different order than those described herein.

Referring to FIG. 3, an example computing device 300 upon which the methods described herein may be implemented is illustrated. It should be understood that the example computing device 300 is only one example of a suitable computing environment upon which the methods described herein may be implemented. Optionally, the computing device 300 can be a well-known computing system including, but not limited to, personal computers, servers, handheld or laptop devices, multiprocessor systems, microprocessor-based systems, network personal computers (PCs), minicomputers, mainframe computers, embedded systems, and/or distributed computing environments including a plurality of any of the above systems or devices. Distributed computing environments enable remote computing devices, which are connected to a communication network or other data transmission medium, to perform various tasks. In the distributed computing environment, the program modules, applications, and other data may be stored on local and/or remote computer storage media.

In its most basic configuration, computing device 300 typically includes at least one processing unit 306 and system memory 304. Depending on the exact configuration and type of computing device, system memory 304 may be volatile (such as random-access memory (RAM)), non-volatile (such as read-only memory (ROM), flash memory, etc.), or some combination of the two. This most basic configuration is illustrated in FIG. 3 by dashed line 302. The processing unit 306 may be a standard programmable processor that performs arithmetic and logic operations necessary for operation of the computing device 300. The computing device 300 may also include a bus or other communication mechanism for communicating information among various components of the computing device 300.

Computing device 300 may have additional features/functionality. For example, computing device 300 may include additional storage such as removable storage 308 and non-removable storage 310 including, but not limited to, magnetic or optical disks or tapes. Computing device 300 may also contain network connection(s) 316 that allow the device to communicate with other devices. Computing device 300 may also have input device(s) 314 such as a keyboard, mouse, touch screen, etc. Output device(s) 312 such as a display, speakers, printer, etc. may also be included. The additional devices may be connected to the bus in order to facilitate communication of data among the components of the computing device 300. All these devices are well known in the art and need not be discussed at length here.

The processing unit 306 may be configured to execute program code encoded in tangible, computer-readable media. Tangible, computer-readable media refers to any media that is capable of providing data that causes the computing device 300 (i.e., a machine) to operate in a particular fashion. Various computer-readable media may be utilized to provide instructions to the processing unit 306 for execution. Example tangible, computer-readable media may include, but is not limited to, volatile media, non-volatile media, removable media, and non-removable media implemented in any method or technology for storage of information such as computer readable instructions, data structures, program modules or other data. System memory 304, removable storage 308, and non-removable storage 310 are all examples of tangible, computer storage media. Example tangible, computer-readable recording media include, but are not limited to, an integrated circuit (e.g., field-programmable gate array or application-specific IC), a hard disk, an optical disk, a magneto-optical disk, a floppy disk, a magnetic tape, a holographic storage medium, a solid-state device, RAM, ROM, electrically erasable program read-only memory (EEPROM), flash memory or other memory technology, CD-ROM, digital versatile disks (DVD) or other optical storage, magnetic cassettes, magnetic tape, magnetic disk storage or other magnetic storage devices.

In an example implementation, the processing unit 306 may execute program code stored in the system memory 304. For example, the bus may carry data to the system memory 304, from which the processing unit 306 receives and executes instructions. The data received by the system memory 304 may optionally be stored on the removable storage 308 or the non-removable storage 310 before or after execution by the processing unit 306.

It should be understood that the various techniques described herein may be implemented in connection with hardware or software or, where appropriate, with a combination thereof. Thus, the methods and apparatuses of the presently disclosed subject matter, or certain aspects or portions thereof, may take the form of program code (i.e., instructions) embodied in tangible media, such as floppy diskettes, CD-ROMs, hard drives, or any other machine-readable storage medium wherein, when the program code is loaded into and executed by a machine, such as a computing device, the machine becomes an apparatus for practicing the presently disclosed subject matter. In the case of program code execution on programmable computers, the computing device generally includes a processor, a storage medium readable by the processor (including volatile and non-volatile memory and/or storage elements), at least one input device, and at least one output device. One or more programs may implement or utilize the processes described in connection with the presently disclosed subject matter, for example, through the use of an application programming interface (API), reusable controls, or the like. Such programs may be implemented in a high-level procedural or object-oriented programming language to communicate with a computer system. However, the program(s) can be implemented in assembly or machine language, if desired. In any case, the language may be a compiled or interpreted language and it may be combined with hardware implementations.

### Artificial Radiotherapy Environment (ARTE)

Radiation damages both cancer cells and normal tissue cells. To quantify the relationship between the applied radiation and the treatment response, the radiation absorbed by tumor and surrounding normal tissue, and the probabilities of tumor control (TCP) and normal tissue complication (NTCP) are considered.

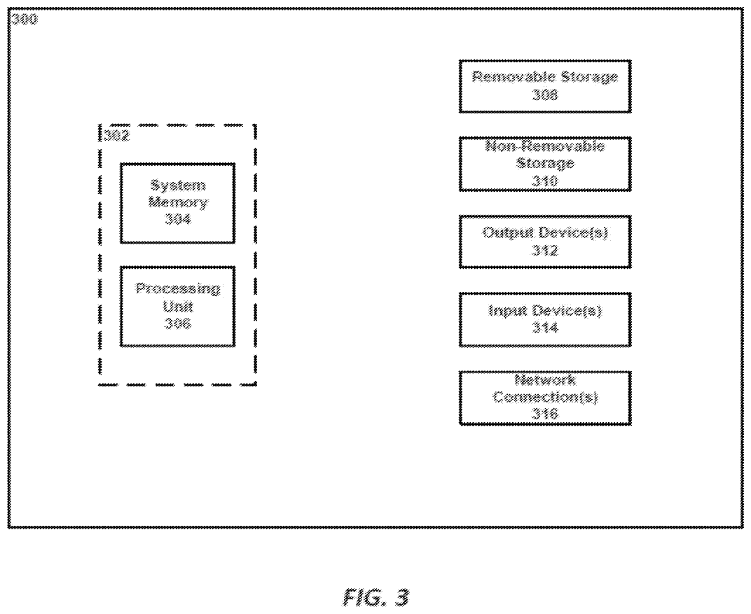

Referring to FIG. 4A, a graph depicting a transition function for gEUD in accordance with certain embodiments of the present disclosure is provided. As shown, the KBRART regimen is divided into three time points, pre, mid, and post treatment, denoted by the daily dose fraction, d, number N₀, Nₑᵥₐₗ, and N_{adapt}, respectively. The treatment period between N₀ and Nₑᵥₐₗ is the Evaluation Phase and between Nₑᵥₐₗ and N_{adapt} is the Adaptation Phase.

The absorbed radiation is spatially non-uniform, so it is generally converted to a homogeneous dose value by weighted-averaging of the treatment sites from treatment planning. Generalized equivalent uniform dose (gEUD) is one such metric [22]. It is expected that for a fixed radiation site, gEUD must increase with increasing applied radiation as shown in FIG. 4A. A linear-quadratic-linear (LOL) type monotonic proportionality relationship was assumed, which further results in the following two relationships for KBR-ART, i.e.,

$$\frac{{g}_{adapt} - {g}_{eval}}{{N}_{adapt} - {N}_{eval}} ∝ \{ \begin{matrix}{d}_{adapt} ( 1 + \frac{{d}_{adapt}}{\frac{α}{β}} ) & for {d}_{adapt} < {D}_{T} \\ {D}_{T} + \frac{{D}_{T}^{2}}{\frac{α}{β}} + ( 1 + \frac{2 {D}_{T}}{\frac{α}{β}} ) ( {d}_{adapt} - {D}_{T} ) & for {d}_{adapt} \geq {D}_{T}\end{matrix} \qquad ( 1 )$$

$$And , \begin{matrix}\frac{{g}_{eval} - {g}_{0}}{{N}_{eval} - {N}_{0}} ∝ \{ \begin{matrix}{d}_{eval} ( 1 + \frac{{d}_{eval}}{\frac{α}{β}} ) & for {d}_{eval} < {D}_{T} \\ {D}_{T} + \frac{{D}_{T}^{2}}{\frac{α}{β}} + ( 1 + \frac{2 {D}_{T}}{\frac{α}{β}} ) ( {d}_{eval} - {D}_{T} ) & for {d}_{eval} \geq {D}_{T}\end{matrix} & ( 2 )\end{matrix}$$

where, g stands for gEUD, N for nth daily dose fractions, d for dose fractions, D_{T} for threshold doses, and α/β ratio is a tissue-specific parameter. The subscript 0, eval, and adapt of N and g corresponds to pre-, mid-, and after-treatment, respectively while dₑᵥₐₗ and d_{adapt} corresponds to applied daily dose fractionations during the evaluation phase and adaptive phase, respectively. Dividing the relationships (1) and (2) yields four equations for g_{adapt} as listed in FIG. 4B which is a table showing Transition Function for final gEUD (i.e., g_{adapt})

With the assumption that the increment of radiation increases both TCP and NTCP, a sigmoid shape generalized logistic function was applied to represent the outcome probability as follows,

$$p = \frac{1}{1 + exp ( \frac{g - μ ( s )}{T ( s )} )} \qquad ( 3 )$$

where the patient-specific parameters u and T are functions of their multiomics state. By applying patient's pre and mid treatment multi-omics information, the above dose-response relationship captures inter-patient heterogeneity.

Applying the equations from FIG. 4B and Eq. (3), ARTE is built as a Markov Decision Process (MDP) as shown in FIG. 2B. ARTE takes in patient's state (s, c) and daily dose fractionation (d) as the input and returns patient's next state (s′) and outcome (tcp, ntcp) as the output. The state dynamics is modeled by the TF and the associated outcome by the RTOE.

### Patient States

Patient state, S⊂ [symbol] ^{k}, represents a patient's information at a given time. It consists of patient's features such as dosimetric, clinical, radiomics, genomics, and imaging information. MDP assumes that patient's state at time t+1 only depends on patient's state and dose, D⊂ [symbol] , at time t. FIG. 6 depicts a Reward Function 3D Contour Plot. In KBR-ART, three time points are relevant as shown in FIG. 6. The reward function, r=tcp(1−ntcp), smoothly raises toward its maximum value at (tcp, ntcp)=(1, 0). AI's goal is to find doses that will result in the maximum reward. As shown,: (i) pretreatment (t=0), (ii) mid treatment (t=eval), and (iii) post treatment (t=adapt).

Previously, deep regression models [5, 17] were applied to learn state transitions for all the patient features. In this work, however, to reduce modeling error, the non-dosimetric variables are considered at any time points as predictors for the treatment outcome and directly use the clinically measured values. Only the state transition is estimated for dosimetric variables since the treatment outcome largely depends on the radiation. Hence, the patient states are divided into time-varying dosimetric variables, S⊂ [symbol] ^{m}, and a set of other fixed multi-omics covariates, C⊂ [symbol] ^{k-m}. A complete patient's state is given by (s, c)∈S×C.

### Transition Function

TF: S×D→S, predicts the next state, sₜ₊₁ for patients in state, sₜ, under given dose, dₜ₊₁. Two TFs were used for the dosimetric variables, tumor gEUD and normal tissue gEUD. It was empirically found that deep model-based TF for gEUDs does not always maintain the causal monotonic relationship. Thus, the LOL based relationship from Eq. (4) below was applied to guarantee an increasing monotone relationship between the dose applied and dose absorbed as presented in Eq. (1) and Eq. (2).

$$\frac{g ( {N}_{i} ) - g ( {N}_{i - 1} )}{{N}_{i} - {N}_{i - 1}} ∝ \{ \begin{matrix}{d}_{i} ( 1 + \frac{{d}_{i}}{\frac{α}{β}} ) & for {d}_{i} < {D}_{T} \\ {D}_{T} + \frac{{D}_{T}^{2}}{\frac{α}{β}} + ( 1 + \frac{2 {D}_{T}}{\frac{α}{β}} ) ( {d}_{i} - {D}_{T} ) & for {d}_{i} \geq {D}_{T}\end{matrix} \qquad ( 4 )$$

### RT Outcome Estimator (RTOE)

In some implementations, the RTOE is generalized logistic function guided double graph convolutional neural network.

RTOEs, TCP: S×C→[0,1] and NTCP:S×C→[0,1], estimate tcp and ntcp for the patient's state, (sₜ₊₁, c). In this work, GNN was applied as the RTOE as shown in FIG. 5A. FIG. 5A shows a Single GNN 501. The Single GNN 501 has an input layer for graph input, graph convolution layers for graph embedding, a global mean pool layer, and a fully connected classifier layer. Generalized logistic function guided double GNN (GLoGD-GNN) 503 has two Single GNNs fed into a 2-parameter logistic function. GLoGD-GNN 503 takes in gEUD as the argument.

Each patient is assigned with a graph of features and then a binary classification is learned on the graph level. First applied a single GNN was applied for RTOE. While the performance is improved drastically compared to a fully connected classifier, the single GNN was found to not respect the monotonicity between the dose value and the outcome probability. To meet the monotonic relationship, a double GNN architecture was applied with a generalized logistic function named as generalized logistic function guided double GNN (GLoGD-GNN).

FIG. 5B is a is a diagram illustrating example operations for training RTOE as a binary classification in accordance with certain embodiments of the present disclosure. The RTOE estimates treatment outcome (tcp, ntcp) for the predicted s_{adapt} and covariate c as a binary classifier. Here θ represent tunable weights, which is learned by minimizing the binary cross entropy loss function. In the case of graph neural networks, the features are input as a directed graph G(V, E), where the nodes, V, represents the features, X⊂ [symbol] ^{k×1} and edges, E, represents the inter-feature connections. Edges, E, are mathematically represented by adjacency matrix, A⊂ [symbol] ^{k×k}. During feedforward, the signal propagation is multiplied by the adjacency matrix given by Hⁱ=σ(AHⁱ⁻¹θⁱ) for zero bias, where σ is the activation function, θᵢ, is the weight of the ith hidden layer, and Hᵢ₋₁ is the matrix containing i-1st layer embeddings. Notice that multiplying by A preserves the only important inter-feature connections and eliminates computational redundancies; each node embedding is computed only once in contrast to fully connected neural network. For this case, features X is a concatenation of dosimetric variable, s, and other multi-omics covariate, c. Note, each patient is represented by a graph in the feature space and the binary classification is performed in the sample space as a graph classification problem.

FIG. 5C is a directed graph showing the inter-relation between the NSCLC patient's features in accordance with certain embodiments. The nodes, which represent features, are color coded with the number of outgoing relationships. Pre stands for pretreatment observation, RD and slope stands for relative difference and change in feature value between pretreatment and mid-treatment observation, respectively.

FIG. 5D is a directed graph showing the inter-relation between the HCC patient's features in accordance with certain embodiments. The nodes, which represent features, are color coded with the number of outgoing relationships. Pre stands for pretreatment observation, RD and slope stands for relative difference and change in feature value between pre-treatment and mid-treatment observation, respectively.

### Reward Function

Reward function, R:[0,1]×[0,1]→ [symbol] , assigns a value to the (tcp, ntcp) pair. The reward function is selected such that its optimization results in maximal tcp and minimal ntcp. R=tcp(1−ntcp) was adopted as reward function for ARCIiDS. As seen from FIG. 6, it is smallest at the negative outcomes, {(tcp, ntcp)}={(0, 0), (0, 1), (1, 1)}, and largest at the positive outcome, (tcp, ntcp)=(1,0).

Additionally, a goal is defined. By default, the goal can be defined as tcp>50% and ntcp<50%, which rounds to positive outcome. Furthermore, goal based on population endpoints can be added. For NSCLC, a goal of tcp>70% and ntcp<17.2%, [17] and for HCC, tcp>90% and ntcp<25% is added.

Combining the reward and goal, the reward scheme for NSCLC and HCC is defined as following,

$${r}_{NSCLC} = \{ \begin{matrix}R + 2 , & if tcp > 0.7 and ntcp < 0.172 \\ R + 1 , & if tcp > 0.5 and ntcp < 0.5 \\ R , & otherwise\end{matrix}$$

$${r}_{HCC} = \{ \begin{matrix}R + 2 , & if tcp > 0.9 and ntcp < 0.25 \\ R + 1 , & if tcp > 0.5 and ntcp < 0.5 \\ R , & otherwise\end{matrix}$$

### Optimal Decision Maker (ODM)

In some implementations, a deep reinforcement learning algorithm was utilized for training the ODM. ODM is composed of a Q (quality) function, DQN:S×C→ [symbol] ^{d}, and a selector. Given a patient's state sₜ, deep Q-net generates a set of q-values, Q⊂ [symbol] ^{d}, for a range of dose. Q-Net maps k-dimensional state space to d-dimensional action (dose-decision) space. During operation mode, it simply follows greedy policy and selects the dose de having the maximum q-value. For training, a model-based RL paradigm was adopted that is divided into two phases: Planning and Learning, as shown in FIG. 2B. During Planning, an exhaustive search is carried out where all patient's states (sₜ, c) and the range of adaptive dose dₜ are fed into the ARTE and the resulting states sₜ₊₁, and rewards rₜ₊₁ are saved into the Memory 107. During Learning, the DON 105 is trained via double DON algorithms using the Memory 107 as a one-step optimization problem.

Uncertainty Estimate via statistical Ensemble

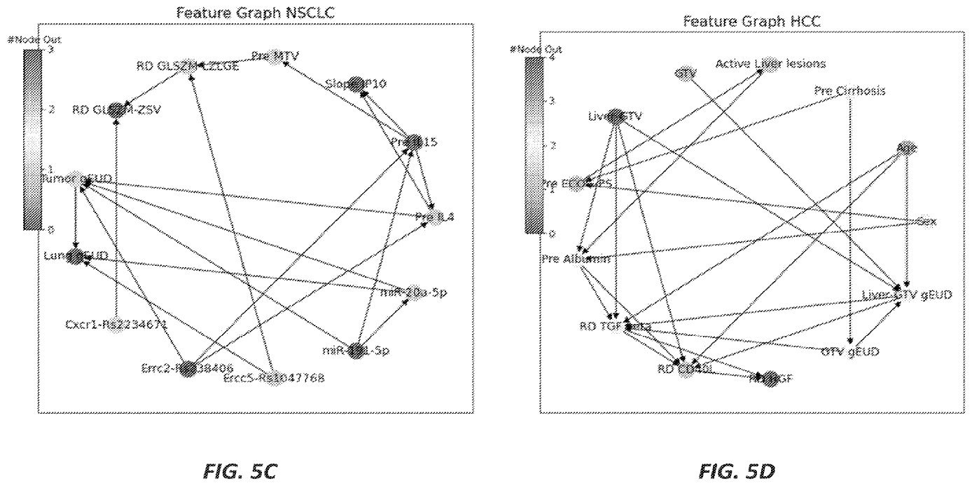

Model uncertainty is estimated using statistical Ensembling. The statistical ensemble technique trains several identical models and finds averages and deviations of the prediction. This method estimates uncertainty purely based on the trained model. Additionally, this also helps with desensitizing ARCIiDS to the noise associated with the stochastic optimization algorithm used by Neural Networks (NNs). NNs utilize a large number of randomly initiated weights and as a result, learned weights are different from model to model ARCIiDS presents the average prediction u as an expected value, and the covariance cov as an uncertainty estimation as shown in FIG. 7. For ODM, standard deviation σ is used as the uncertainty estimate. FIG. 7 is a schematic diagram showing Model Uncertainty via statistical Ensemble. N identical models were trained. The mean and covariance (or standard deviation) of the output distribution captures the model output and model uncertainty. For RTOE's probability outputs, the uncertainty estimates were presented as the covariance and for ODM dose recommendations, the uncertainty estimates were presented as the standard error of mean.

### Analysis

ARCIiDS was trained and validated on two different use cases from two different types of RT treatments. The first use case is an adaptive RT clinical trial of NSCLC patients, and the second use case is an adaptive SBRT clinical trial. After feature selections, five ARTEs were built for each disease using the dataset. Then, 10,000 synthetic patients were generated using a generative adversarial network (GAN)[26]. Five ODMs were trained using the five ARTEs and 4,000 randomly chosen patients from the pool of the 10,000 synthetic patients. The trained ODM models were then validated on the original dataset.

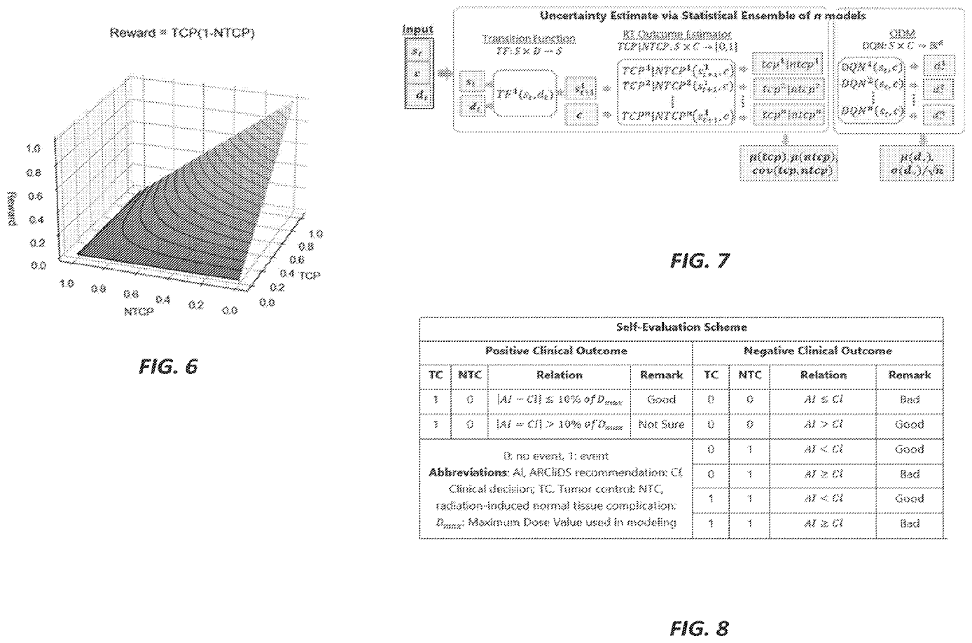

Since there is no ground truth of what an optimal radiation dosage for a certain outcome would be, evaluations are based on two metrics. The first metric is root mean square difference (RMSD) value between the ODM recommendation and the retrospective clinical decision used in treatment planning. However, since RMSD is a symmetric metric, i.e., it cannot differentiate a higher dose from a lower dose recommendation compared to the clinical decision, the positive and negative clinical outcomes were separated for additional insight. For the positive clinical outcome, a lower RMSD indicates agreement with the good clinical decisions. For the bad clinical outcome, additional comparison is needed. For this purpose, a second metric for self-evaluation was adopted as presented in FIG. 8. FIG. 8 is a self-evaluation scheme for AI recommendation based on the positive relation between radiation dose and treatment outcomes, i.e., both TCP and NTCP increases with an increase in radiation dose. Here TC and NTC are clinical treatment outcome. TC=1 and NTC=0 are the only clinically positive outcome. For a patient with a known treatment outcome, an AI recommendation can be evaluated by comparing it with the retrospective clinical decision. For instance, for a patient with TC=0 and NTC=0, a higher dose recommendation is good, while for a patient with TC=1 and NTC=1, a lower dose recommendation is good, and for a patient with TC=0, NTC=1, a lower dose recommendation is good. For the clinically positive cases, it is unclear if a recommendation is good unless it is within a window of the clinical dose decision. The window was set to be 10% of the maximum dose used in the modeling. The self-evaluation scheme is also based on the assumption that increasing radiation results in a higher value for both TCP and NTCP. Using this assumption, the recommendations for patients with negative clinical outcome can be evaluated further.

### Example Graphical User Interface

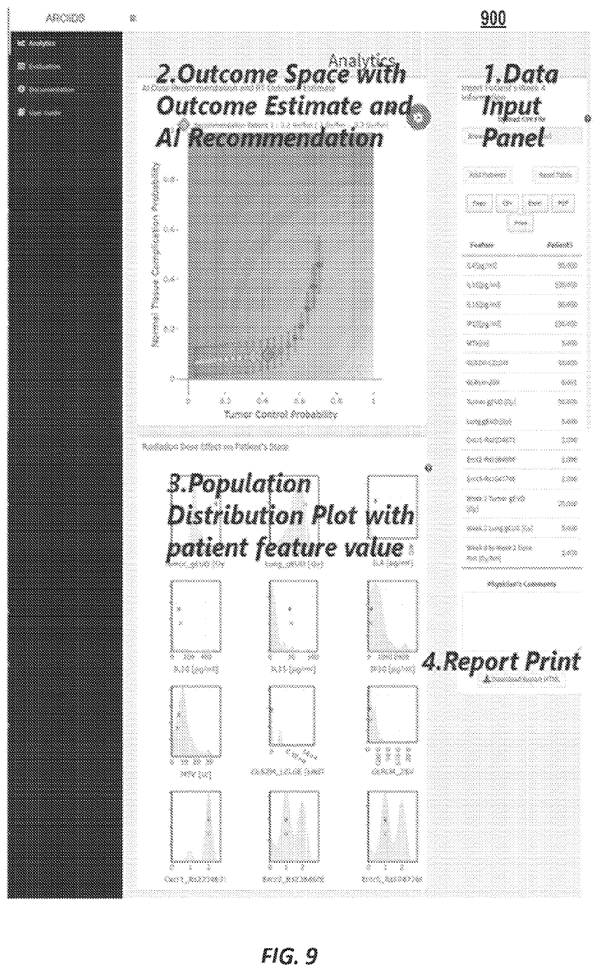

FIG. 9 depicts an ARCIiDS graphical user interface (GUI) 900 in accordance with certain embodiments of the present disclosure. ARCIiDS was designed as a Web Application (app) using R Shiny as shown in FIG. 9. The GUI 900 consists of the Data Input Panel, Outcome Space spanned by TCP and NTCP, historic Population Distribution Plots, and Report Print. The app consists of 4 main panels: Data Input Panel, Outcome Space, Population Distribution Plot and Report Print. Beside these, there are accessibility tools such as help information, user guide, documentation, zooming, and printing option.

### Data Input Panel

Patient Data can be input manually or via a data file. Multiple patients' states can be input for visual comparison. The inputs can be saved or printed if necessary.

There is a dedicated space for Physician notes.

### Outcome Space

The AI recommendations are presented in the Output Space. The Output Space is spanned by TCP in the x-axis and NTCP in the y-axis. It is contoured and colored with the Reward Function, providing additional insight on the AI's Decision Making. Given a patient's information, it shows treatment outcome for a range of daily dose fractions and marks the treatment outcome for the optimal dose recommendation. It provides uncertainty assessment for both the outcome estimate and AI recommendation.

### Population Distribution Plot

Knowing the patient's state value and its relative position to the population provides information on patient's “whereabouts”. To accommodate a comparison on the feature level, histograms are included for each feature and patient's state value atop.

### Report Print

A report printing in html format was designed. The interactive nature of the report, even outside the app, makes it much easier to communicate with other users.

### Results

### Multi-omics Feature Selection for TCP and NTCP

Thirteen important multi-omics features resulted from the multiobjective Markov Blanket feature selection process. These features are important predictors for both TCP and NTCP. For NSCLC, the selected features are cytokines: pretreatment interleukin 4 (pre-IL4), pre-IL15 and slope of Interferon gamma-induced protein 10 (slope-IP10); Tumor PET imaging features/Radiomics: pretreatment Metabolic Tumor Volume (pre-MTV), relative difference (RD) of Gray-level size zone matrices (GLSZM)-large zone low gray-level (LZLGE) and RD-GLSZM-zone size variance (RD-GLSZM-ZSV); Dosimetry: Tumor gEUD and Lung gEUD; Genetics (single nucleotide polymorphism [SNP]): Cxcr1-Rs2234671, Ercc2-Rs238406, and Ercc5-Rs1047768; and MicroRNA: miR-191-5p and miR-20a-5p. For HCC, the selected features are clinical: sex, age, pretreatment cirrhosis status (pre-cirrhosis), pretreatment Eastern Cooperative Oncology Group Performance status (pre-ECOG-PS), number of active liver lesions (active lesions), pretreatment albumin level (pre-albumin); Tumor PET Imaging: gross tumor volume (GTV) and liver volume minus GTV (Liver-GTV); Dosimetry: GTV gEUD and Liver-GTV volume; and cytokines/signaling molecule: relative difference of Transforming growth factor beta (RD-TGF-β), Cluster of Differentiation 40 receptor's Ligand (RD-CG40L), and Hepatocyte growth factor (RD-HGF). Here the slope and RD were determined from comparing pretreatment and mid-treatment or the end of evaluation phase.

ARTE with GLoGD-GNN Architecture Yielded Expected Monotonic Dose-Response

As expected, correction of RTOE with GLoGD architecture helps to maintain the monotonic relationship between the outcome probability and daily dose fractionation. The area under the receiver operating characteristics curve (AUROCC) analysis was performed to measure the performance of single GNN and GLoGD GNN architecture. For the analysis, the data set was split via a 10-fold stratified shuffle 80-20 split process. For NSCLC, the single-GNN AUROCC for TCP and NTCP were 0.77±0.14 (mean±SD) and 0.73±0.18, while GLoGD-AUROCC for TCP and NTCP were 0.73±0.15 (mean±SD) and 0.79±0.17, respectively. For HCC, the single-GNN AUROCC for TCP and NTCP were 0.72±0.31 (mean±SD) and 0.81±0.14, while GLoGD-AUROCC for TCP and NTCP were 0.74±0.27 (mean±SD) and 0.68±0.24, respectively. The performance of GLoGD-GNN on the population dataset is either similar or poor compared to single-GNN because GLoGD-GNN is designed for individual patients.

Generating Synthetic Patients via GAN for training ODM

To extend the sample size of the dataset for training ODM, a Wasserstein Generative Adversarial Network with Gradient Penalty (WGAN-GP) 5,26 was trained on the original dataset and generated 10,000 synthetic patients. GAN can learn the underlying population distribution in the multi-dimensional feature space. The distribution of synthetic patient was compared with the original patient population data using the Jensen Shannon Divergence (JSD) metric. JSD value of 0 means complete overlap and 1 means complete separation. JSD for NSCLC's selected features were pre-IL4: 0.30, pre-IL15: 0.18, slope-IP10: 0.50, Pre-MTV: 0.46, RD-GLSZM-LZLGE: 0.59, RD-GLSZM-ZSV: 0.56, Tumor-gEUD: 0.60, LunggEUD: 0.43, miR-191-5p: 0.18, miR-20a-5p: 0.18, cxcr1-Rs2234671: 0.16, errc2-Rs238406: 0.24, and ercc5-Rs1047768: 0.07. JSD for HCC's selected features were sex: 0.03, age: 0.44, precirrhosis: 0.25, active lesion: 0.31, GTV: 0.33, Liver-GTV: 0.36, pre-ECOG-PS: 0.16, pre-albumin: 0.46, RD-TGF-β: 0.47, RD-CD40L: 0.45, RD-HGF: 0.75, GTV-gEUD: 0.45, and Liver-GTV gEUD: 0.45. Note that, no statistical hypothesis test was performed on the learned distribution as it is not necessary for the training of ODM. In principle, a uniform distribution works just as fine, however, with increase in computational complexity. Nevertheless, the similarity was additionally ascertained by visual inspection.

### RMSD Evaluation of ARCIiDS Recommendation

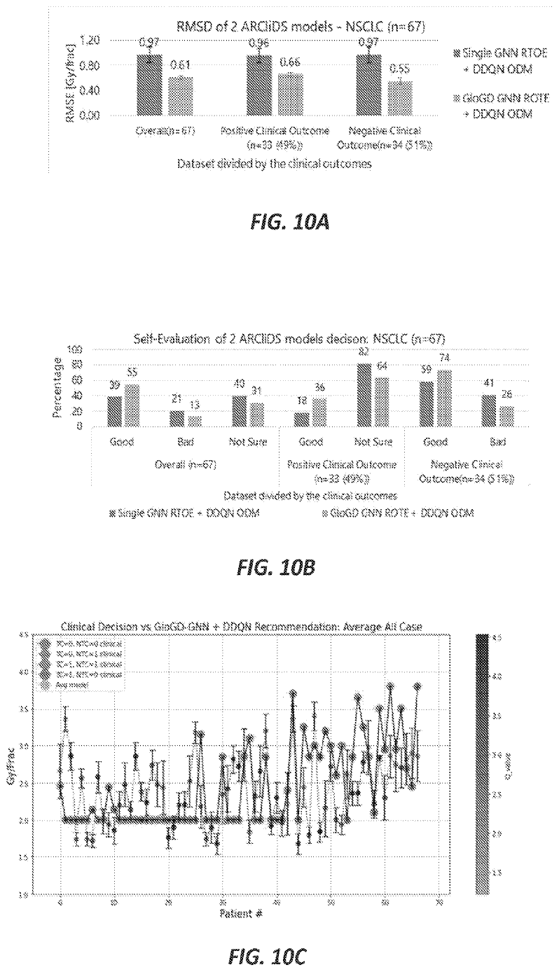

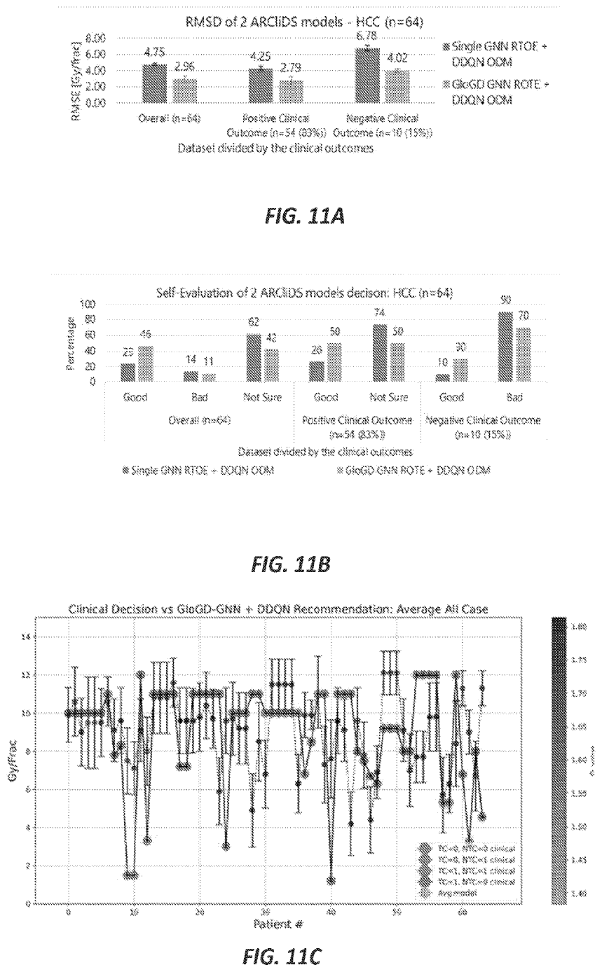

The ODM was trained on the synthetic dataset using the trained ARTE and validated on the original dataset. The main results are summarized and presented in FIGS. 10A-10C and FIG. 11A-11C. For the NSCLC patients, the overall RMSDs between the two ARCIiDS models' average recommendation and reported clinical decisions, ordered as GLoGD GNN RTOE+DDQN ODM and Single GNN ROTE+DDQN ODM, were 0.61±0.03 Gray/fraction [Gy/frac] (mean±sem) vs 0.97±0.12 Gy/frac, respectively. The RMSDs for patients with positive clinical outcomes were 0.66±0.02 Gy/frac vs 0.96±0.11 Gy/frac respectively, and for patients with negative clinical outcomes were 0.55±0.05 Gy/frac vs 0.97±0.12 Gy/frac, respectively.

For the HCC patients, the overall RMSDs were 2.96±0.42 Gy/frac vs 4.75±0.16 Gy/Frac, respectively. The RMSD for patients with positive clinical outcomes were 2.79±0.50 Gy/frac vs 4.25±0.26 Gy/frac, respectively, and for patients with negative clinical outcomes were 4.02±0.23 Gy/frac vs 6.78±0.35 Gy/frac, respectively.

### Self-Evaluation of ARCIiDS Recommendation

For the NSCLC patients, the overall Self-Evaluation results between the two ARCIiDS models' average recommendation and reported clinical decisions, ordered as GLoGD GNN RTOE+DDQN ODM and Single GNN ROTE+DDQN ODM, were Good: 55% vs 39%, Bad: 13% vs 21%, and Not Sure: 13% vs 40%, respectively. The Self-Evaluation results for patients with positive clinical outcomes, were Good: 36% vs 18%, and Not Sure: 82% vs 64%, respectively, and for patients with negative clinical outcomes were Good: 74% vs 59%, and Bad: 26% vs 41%, respectively.

For the HCC patients, the overall Self-Evaluation results were Good: 46% vs 23%, Bad: 11% vs 14%, and Not Sure: 42% vs 62%, respectively. The Self-Evaluation results for patients with positive clinical outcomes were Good: 50% vs 26%, and Not Sure: 50% vs 74%, respectively., and for patients with negative clinical outcomes were Good: 30% vs 10%, and Bad: 70% vs 90%, respectively.

A comparison for two different ARCIiDS models is provided below. The first is built with Single GNN as RTOE and fully connected double deep Q-network as ODM (Single GNN RTOE+DDQN ODM) and the second with GLoGD GNN as RTOE and fully connected double deep Q-network as ODM (GLoGD GNN RTOE+DDQN ODM).

FIG. 10A, FIG. 10B, and FIG. 10C are graphs showing a comparison and analysis of two ARCIiDS models trained and validated on Adaptive RT NSCLC patients. FIG. 10A presents RMSD of the 2 ARCIiDS models, and FIG. 10B presents Self-Evaluation of the 2 ARCIiDS models, and FIG. 10C is a graph presenting a visual comparison between the ARCIiDS recommendation and clinical decision. The clinical decisions are color coded with the outcomes and the ARCIiDS recommendations are color coded with the respective q-value. Qualitatively, the q-value can be considered as the AI confidence in its recommendations.

FIG. 11A, FIG. 11B, and FIG. 11C are graphs showing comparison and analysis of two ARCIiDS models trained and validated on Adaptive RT HCC patients. FIG. 11A presents RMSD of the 2 ARCIiDS models, FIG. 11B presents Self-Evaluation of the 2 ARCIiDS models, and FIG. 11C presents a visual comparison between the ARCIiDS recommendation and clinical decision. The clinical decisions are color coded with the outcomes and the ARCIiDS recommendations are color coded with the respective q-value. Qualitatively, the q-value can be considered as the AI confidence in its recommendations.

### DISCUSSION

To our knowledge, there are software for ART 27,28 but ARCIiDS is the first interactive software dedicated to KBR-ART that will be available through a web portal. This disclosure demonstrates its applicability to adaptive RT and SBRT. However, ARCIiD's underlying technology can be generalized to any other DTR to optimize sequential decision-making with multi-omics data for deciding the order of treatments, including multi-modality treatment, given that an artificial treatment environment can be sufficiently modeled.

Embodiments of the present disclosure implement tools such as GAN and GNN and invented novel techniques such as GLoGD-GNN to overcome data-related issues for developing ARCIiDS. GAN was applied to learn the underlying patient's feature distribution and generated 10,000 synthetic patients for training the ODM. GNN was adopted for modeling RTOE as exploiting the inter-relationship between the features can improve model prediction. Mathematically, the inter-relationship can be represented by a directed graph G(V,E) where the nodes V represent patient features and edges E represent the relationships. Analyzing the interfeature relationships before feeding it to the NN reduces the number of connections and hence simplify the learning process. As a novel approach, GNN was applied on the feature space as opposed to the sample space. As shown in the SM, every patient is represented by a directed graph of features, set by the treatment and disease type. RTOE is then designed as a graph classification problem where the node value differs from patient to patient.

As seen from FIGS. 10A-C and FIG. 11A-C, the models in descending order according to the RMSD and Self-Evaluation measures, for both NSCLC and HCC, are GLoGD GNN RTOE+DDQN ODM, and Single GNN RTOE+DDQN ODM. As expected, correction of RTOE with GLoGD architecture helps to maintain the monotonic relationship between the outcome probability and daily dose fractionation and in turn helped ARCIiDS in making better recommendation.

The framework described herein has some limitations. Clinically, RT dose adaptation can be performed in different ways: (1) change dose per fractions, and (2) change the number of fractions. For SBRT, the former is suitable, however for some diseases and modalities the latter may be more appropriate. For instance, when RT is combined with chemotherapy, increasing the number of fractions is preferred. The framework only covers the former. ARCIiDS uses several biomarkers such as cytokines as predictors. Due to the lack of standardization, biomarker levels of the same blood sample measured in two labs can be quite different, also known as batch effect. So, biomarker levels of external dataset must be carefully examined before applying ARCIiDS. For dosimetric predictor, gEUD was used, however, for lung and liver, mean dose could also be applied. Another limitation is the number of NTCP's considered in ARCIiDS. In practice there may be more than 1 normal tissues of interest. For NSCLC, heart and lungs are the dose-limiting organs at risk (OAR). For HCC, although liver is usually the main OAR, in some patient, who has tumors near the intestine, intestine is also considered during designing the treatment plan. Finally, beside data-related shortcomings, ARCIiDS prediction and recommendation uncertainty, which is based on statistical ensembles, can be improved by training more models; however, this will require more computational power and time.

Although the largest dataset of its kind was utilized, a larger sample size and balanced dataset will improve ARCIiDS performance. The subsequent paragraphs discuss data-related limitations, methods implemented to overcome those limitations, and other possible solutions.

The learning of an environment model is the bottle neck of ARCIiDS. For learning a good ARTE, a sufficient sample size and a balanced dataset are necessary. In the adaptive HCC patient's cohort, only 1 patient did not achieve local control. As a result, RTOE for TCP had an unusually high AUROCC uncertainty. Although class-imbalance correction techniques such as SMOTE and weighted loss function were applied, it was observed that such techniques fall short in correcting a highly imbalanced dataset. In addition, the toxicity count was also low-—there were only 7 patients that showed toxicity. While this is a clinically desirable result, it hinders the learning process and hampers model generalizability. To make the matter worse, patients with highest liver gEUD didn't show toxicity. This reflects interpatient heterogeneity, where some patients have poor pre-treatment liver function, who at a higher risk of toxicity for lower dose. Nevertheless, a hyperparameter search for maximizing the generalizability of ARTE was performed.

High noise-to-signal ratio due to inter-patient heterogeneity becomes even more pronounced with a small sample size. The medical field is especially doomed with a small sample size primarily due to the privacy issue [29]. Such issues make it difficult to learn correct trends in purely data-driven learning. It was found that Single-GNN RTOE predicted unphysical trends between daily dose fractionation and TCP/NTCP. For correction, a GLoGDGNN architecture was applied to infuse prior knowledge into the data-driven technique. It was found that it corrects the trend and can also increase the model predictability. Alternatively, distributive learning features such as federated learning can be added to ARCIiDS to overcome the small sample size issue. In federated learning approach only the model parameters are shared and data stays within the firewall of individual institutions30.

Sample size issue in training ODM can also be overcome by using synthetic patients. Since ODM of ARCIiDS learns via model-based reinforcement learning, computationally the task of ODM is to learn ARTE. This can be considered as an interpolation problem in a continuous feature space. This problem can be tackled using brute-force by exhaustively selecting patient's state. However, this assumes a uniform distribution which is generally not true. Therefore, generative adversarial network (GAN) was applied to learn the underlying patient's feature distribution and generated synthetic patient states for training the ODM. In principle, a conditional GAN31 can be applied to generate patients states distribution along with the outcome, however, a low sample size coupled with severe class-imbalance makes it impossible to correctly learn the underlying conditional probability distribution.

The RMSD values for adaptive SBRT in HCC were higher than adaptive RT in NSCLC. There are three reasons for the higher RMSD value: (1) A larger range of adaptive dose values was explored for SBRT, i.e., 1 to 15 Gy/frac compared to 1.5 to 4 Gy/frac; given that the sample sizes are comparable, the datapoints for HCC are much sparser resulting in higher interpolation error; (2) most of the patients with a clinically negative outcome for SBRT received a lower adaptive dose than the positive case; this can confuse the RTOE, which assumes higher doses results in higher TCP and NTCP; (3) due to class-imbalance, the corrected GLoGD-GNN RTOE yielded a flatter monotonic relation than expected that did not spanned the whole probability space; it was observed that the AI agent failed to satisfy the population-based goal of TCP>90% and NTCP<25%. So, the computation goal of TCP>50% and NTCP<50% was set. A smaller RMSD value can be achieved with a large sample size and well-balanced dataset.

In conclusion, embodiments of the present disclosure provide a userfriendly software for AI-assisted clinical decision-making and demonstrate its performance in adaptive RT. The underlying technology behind the software is generalizable to other sequential decision-making tasks in oncology. GNNs were employed to exploit the inter-feature relationship. The software was trained and validated in two different treatment types for two different diseases. The training and validation were repeated for two different models to test our hypothesis of improved model performance. The results confirmed our hypothesis. statistical Ensemble was adopted to assess the model uncertainty.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

The following patents, applications, and publications, as listed below and throughout this document, are hereby incorporated by reference in their entirety herein.
1. Leech, M., Katz, M. S., Kazmierska, J., McCrossin, J. & Turner, S. Empowering patients in decision-making in radiation oncology—can we do better? Mol Oncol 14, 1442-1460 (2020).
2. Niraula, D. et al. Current status and future developments in predicting outcomes in radiation oncology. Br J Radiol (2022) doi: 10.1259/bjr.20220239.
3. Naqa, I. el, Kosorok, M. R., Jin, J., Mierzwa, M. & ten Haken, R. K. Prospects and challenges for clinical decision support in the era of big data. JCO Clin Cancer Inform 2, (2018).
4. Tseng, H.-H., Luo, Y., ten Haken, R. K. & el Naqa, I. The Role of Machine Learning in Knowledge-Based Response-Adapted Radiotherapy. Front Oncol 8, (2018).
5. Niraula, D., Jamaluddin, J., Matuszak, M. M., Haken, R. K. ten & Naqa, I. el. Quantum deep reinforcement learning for clinical decision support in oncology: application to adaptive radiotherapy. Sci Rep 11, 23545 (2021).
6. Sun, W. et al. Precision radiotherapy via information integration of expert human knowledge and AI recommendation to optimize clinical decision making. Comput Methods Programs Biomed 221, 106927 (2022).
7. Adaptive Treatment strategies in Practice. (Society for Industrial and Applied Mathematics, 2015). doi: 10.1137/1.9781611974188.
8. Chakraborty, B. & Murphy, S. A. Dynamic Treatment Regimes. Annu Rev stat Appl 1, 447-464 (2014).
9. el Naqa, I. et al. Radiogenomics and radiotherapy response modeling. Phys Med Biol 62, R179-R206 (2017).
10. el Naqa, I. et al. Radiation Therapy Outcomes Models in the Era of Radiomics and Radiogenomics: Uncertainties and Validation. International Journal of Radiation Oncology*Biology*Physics 102, 1070-1073 (2018).
11. Kamran, S. C. & Mouw, K. W. Applying Precision Oncology Principles in Radiation Oncology. JCO Precis Oncol 1-23 (2018) doi: 10.1200/PO.18.00034.
12. Glide-Hursₜ, C. K. et al. Adaptive Radiation Therapy (ART) strategies and Technical Considerations: A state of the ART Review From NRG Oncology. International Journal of Radiation Oncology*Biology*Physics 109, 1054-1075 (2021).
13. Kong, F.-M. et al. Effect of Midtreatment PET/CT-Adapted Radiation Therapy With Concurrent Chemotherapy in Patients With Locally Advanced Non-Small-Cell Lung Cancer. JAMA Oncol 3, 1358 (2017).
14. Jackson, W. C. et al. A mid-treatment break and reassessment maintains tumor control and reduces toxicity in patients with hepatocellular carcinoma treated with stereotactic body radiation therapy. Radiotherapy and Oncology 141, 101-107 (2019).
15. USFDA. Artificial Intelligence/Machine Learning (AI/ML)-Based Software as a Medical Device (SaMD) Action Plan. https://www.fda.gov/media/145022/download (2021).
16. USFDA. Proposed Regulatory Framework for Modifications to Artificial Intelligience/Machine Learning (AI/ML)-Based Software as a Medical Device (SaMD)). https://www.fda.gov/media/122535/download (2019).
17. Tseng, H.-H. et al. Deep reinforcement learning for automated radiation adaptation in lung cancer. Med Phys 44, 6690-6705 (2017).
18. Goodfellow, I., Bengio, Y. & Courville, A. Deep Learning. (MIT Press, 2016).
19. Hamilton, W. L. Graph Representation Learning. vol. 14 (Morgan and Claypool, 2020).
20. Luo, Y. et al. A multiobjective Bayesian networks approach for joint prediction of tumor local control and radiation pneumonitis in nonsmall-cell lung cancer (NSCLC) for response-adapted radiotherapy. Med Phys 45, 3980-3995 (2018).
21. Sutton, R. S. & Barto, A. G. Reinforcement Learning: An Introduction. (MIT Press, 2018).
22. Allen Li, X. et al. The use and QA of biologically related models for treatment planning: Short report of the TG-166 of the therapy physics committee of the AAPM. Med Phys 39, 1386-1409 (2012).
23. A Guide to Outcome Modeling In Radiotherapy and Oncology: Listening to the Data. (CRC Press, 2018).
24. Hasselt, H. van, Guez, A. & Silver, D. Deep Reinforcement Learning with Double Q-Learning. in Proceedings of the Thirtieth AAAI Conference on Artificial Intelligence 2094-2100 (AAAI Press, 2016).
25. Roberts, D. A., Yaida, S. & Hanin, B. The Principles of Deep Learning Theory. (Cambridge University Press, 2022).
26. Gulrajani, I., Ahmed, F., Arjovsky, M., Dumoulin, V. & Courville, A. Improved Training of Wasserstein GANs. (2017).
27. Sousa, F. et al. Re-planning assessment in head and neck cancer radiotherapy: 3 years single institution experience. in Re-planning assessment in head and neck cancer radiotherapy: 3 years single institution experience (ESTRO 2022, 2022).
28. Gros, S. A. A. et al. Retrospective Clinical Evaluation of a Decision-Support Software for Adaptive Radiotherapy of Head and Neck Cancer Patients. Front Oncol 12, (2022).
29. Beyond the HIPAA Privacy Rule. (National Academies Press, 2009). doi: 10.17226/12458.
30. Topaloglu, M. Y., Morrell, E. M., Rajendran, S. & Topaloglu, U. In the Pursuit of Privacy: The Promises and Predicaments of Federated Learning in Healthcare. Front Artif Intell 4, (2021).
31. Mirza, M. & Osindero, S. Conditional Generative Adversarial Nets. (2014).
1.* Luo, Y. et al. A multiobjective Bayesian networks approach for joint prediction of tumor local control and radiation pneumonitis in nonsmall-cell lung cancer (NSCLC) for response-adapted radiotherapy. Med Phys 45, 3980-3995 (2018).
2.* Hamilton, W. L. Graph Representation Learning. vol. 14 (Morgan and Claypool, 2020).
3.* A Guide to Outcome Modeling In Radiotherapy and Oncology: Listening to the Data. (CRC Press, 2018).
4. Ramírez, M. F., Huitink, J. M. & Cata, J. P. Perioperative Clinical Interventions That Modify the Immune Response in Cancer Patients. Open J Anesthesiol 03, 133-139 (2013).
5.* Schaue, D., Kachikwu, E. L. & McBride, W. H. Cytokines in Radiobiological Responses: A Review. Radiat Res 178, 505-523 (2012).
6.* Warltier, D. C., Laffey, J. G., Boylan, J. F. & Cheng, D. C. H. The Systemic Inflammatory Response to Cardiac Surgery. Anesthesiology 97, 215-252 (2002).
7.* Mahasittiwat, P. et al. Metabolic tumor volume on PET reduced more than gross tumor volume on CT during radiotherapy in patients with non-small cell lung cancer treated with 3DCRT or SBRT. J Radiat Oncol 2, 191-202 (2013).
8.* Carrier-Vallieres, M. Radiomics: enabling factors towards precision medicine. (McGill University, 2018).
9.* A Guide to Outcome Modeling In Radiotherapy and Oncology: Listening to the Data. (CRC Press, 2018).
10.* Hildebrandt, M. A. T. et al. Genetic Variants in Inflammation-Related Genes Are Associated with Radiation-Induced Toxicity Following Treatment for Non-Small Cell Lung Cancer. PLoS One 5, e12402 (2010).
11.* Chang, J. S. et al. Nucleotide excision repair genes and risk of lung cancer among San Francisco Bay Area Latinos and African Americans. Int J Cancer 123, 2095-2104 (2008).
12.* Kiyohara, C. & Yoshimasu, K. Genetic polymorphisms in the nucleotide excision repair pathway and lung cancer risk: A meta-analysis. Int J Med Sci 59-71 (2007) doi: 10.7150/ijms.4.59.
13.* Nagpal, N. & Kulshreshtha, R. miR-191: an emerging player in disease biology. Front Genet 5, (2014).
14.* Ricciuti, B. et al. Non-coding RNAs in lung cancer. Oncoscience 1, 674-705 (2014).
15.* Lin, S. & Gregory, R. I. MicroRNA biogenesis pathways in cancer. Nat Rev Cancer 15, 321-333 (2015).
16.* Caraceni, P., Tufoni, M. & Bonavita, M. E. Clinical use of albumin. Blood Transfus 11 Suppl 4, s18-25 (2013).
17.* Morikawa, M., Derynck, R. & Miyazono, K. TGF-β and the TGF-β Family: ContextDependent Roles in Cell and Tissue Physiology. Cold Spring Harb Perspect Biol 8, (2016).
18.* Elgueta, R. et al. Molecular mechanism and function of CD40/CD40L engagement in the immune system. Immunol Rev 229, 152-172 (2009).
19.* Perreau, M. et al. The cytokines HGF and CXCL13 predict the severity and the mortality in COVID-19 patients. Nat Commun 12, 4888 (2021).
20.* Matsumoto, K. & Nakamura, T. Roles of HGF as a pleiotropic factor in organ regeneration. EXS 65, 225-49 (1993).

1. A supervised machine learning model training method, comprising:
providing an artificial radiotherapy environment model comprising:
a transition function model configured to predict a next state based on a given state and a given radiation dose for a patient,
a radiotherapy outcome estimator model configured to predict a treatment outcome for the next state, the radiotherapy outcome estimator model comprising at least two artificial neural networks and a logistic function, wherein respective outputs of the at least two artificial neural networks are fed into the logistic function, and
a reward function configured to assign a reward for the next state based on the treatment outcome for the next state; and
training the artificial radiotherapy environment model with a labeled dataset comprising a plurality of patient records, each patient record comprising respective patient information and a respective retrospective dose plan, wherein the trained artificial radiotherapy environment model is configured to output, for the given state and the given radiation dose, the next state, the treatment outcome for the next state, and the reward for the next state.

2. The method of claim 1, further comprising imposing prior knowledge on at least one feature of the next state for the patient as output by the transition function of the transition function model using a model for radiotherapy.

3. The method of claim 2, wherein the model for radiotherapy is a linearquadratic-linear (LQL) model.

4. The method of claim 1, wherein each of the at least two artificial neural networks of the radiotherapy outcome estimator model is tuned individually with other artificial neural networks of the at least two artificial neural networks fixed.

5. The method of claim 4, wherein each of the at least two artificial neural networks of the radiotherapy outcome estimator model is a graph convolutional neural network (GNN).

6. The method of claim 1, wherein the radiotherapy outcome estimator model is a generalized logistic function guided double graph convolutional neural network.

7. The method of claim 1, wherein the reward function is a function of tumor control probability and normal tissue complication probability where optimizing the reward function maximizes the tumor control probability while minimizing the normal tissue complication probability.

8. The method of claim 1, wherein the given state and the next state comprise one or more-multi-omic features.

9. The method of claim 1, wherein the given state and the next state comprise one or more of genomic, radiomic, proteomic, dosimetric, and metabolic tumor volume features.

10. The method of claim 1, wherein the treatment outcome for the next state comprises a probability of tumor local control and a probability of radiation-induced normal tissue complication.

11. A reinforcement learning model training method, comprising:
providing an optimal decision-maker model, wherein the optimal decision-maker model comprises a deep reinforcement learning model; and
providing an artificial radiotherapy environment model, the artificial radiotherapy environment model comprising:
a transition function model configured to predict a next state based on a given state and a given radiation dose for a patient,
a radiotherapy outcome estimator model configured to predict a treatment outcome for the next state, the radiotherapy outcome estimator model comprising at least two artificial neural networks and a logistic function, wherein respective outputs of the at least two artificial neural networks are fed into the logistic function, and
a reward function configured to assign a reward for the next state based on the treatment outcome for the next state;
training, using the trained artificial radiotherapy environment model, a deep reinforcement learning model with a labeled dataset comprising a plurality of patient records, each patient record comprising respective patient information and a respective retrospective dose plan, wherein the trained artificial radiotherapy environment model is configured to output, for the given state and the given radiation dose, the next state, the treatment outcome for the next state, and the reward for the next state, wherein the trained deep reinforcement learning model is configured to predict an optimal dose for the patient.

12. The method of claim 11, wherein the optimal dose for the patient maximizes tumor local control and/or minimizes radiation-induced normal tissue complications.

13. The method of claim 11, wherein the deep reinforcement learning model is a double-Q learning model.

14. The method of claim 13, wherein the double-Q learning model is trained using a planning and learning scheme.

15. A method for providing adaptive radiotherapy clinical decision support, the method comprising:
providing an artificial radiotherapy environment model, the artificial radiotherapy environment model comprising:
a transition function model configured to predict a next state based on a given state and a given radiation dose for a patient,
a radiotherapy outcome estimator model configured to predict a treatment outcome for the next state, the radiotherapy outcome estimator model comprising at least two artificial neural networks and a logistic function, wherein respective outputs of the at least two artificial neural networks are fed into the logistic function, and
a reward function configured to assign a reward for the next state based on the treatment outcome for the next state;
training, using the trained artificial radiotherapy environment model, a
deep reinforcement learning model with a labeled dataset comprising a plurality of patient records, each patient record comprising respective patient information and a respective retrospective dose plan, wherein the trained artificial radiotherapy environment model is configured to output, for the given state and the given radiation dose, the next state, the treatment outcome for the next state, and the reward for the next state;
receiving a current state for a new patient;
inputting the current state into the trained deep reinforcement learning model; and
predicting, using the trained deep reinforcement learning model, an optimal treatment dose for the new patient.

16. The method of claim 15, further comprising providing the optimal treatment dose for the new patient.

17. The method of claim 15, further comprising providing an uncertainty estimate, wherein the uncertainty estimate is related to an output of the trained artificial radiotherapy environment model.

18. (canceled)

19. The method of claim 17, wherein the uncertainty estimate is based on a statistical ensemble or related to an output of the trained deep reinforcement learning model.

20. (canceled)

21. (canceled)

22. The method of claim 15, further comprising: collecting confidence data related to the trained deep reinforcement learning model from a plurality of users.

23. (canceled)

24. The method of claim 22, wherein the confidence data is collected during blind or seen interactions with the plurality of users.
